(19) United States
(12) Patent Application Publication
(10) Pub. No.: US 2006/0008464 A1
(43) Pub. Date: Jan. 12, 2006

(54) Histone conjugates and uses thereof

(76) Inventors: Chaim Gilon, Jerusalem (IL); Abraham Loyter, Jerusalem (IL); Adolf Graessman, Berlin (DE); Ilana Hariton-Gazal, Rehovot (IL); Joseph Sperling, Jerusalem (IL)

Correspondence Address: Martin Moynihan;Anthony Castorina, Suite 207, 2001 Jefferson Davis Highway, Arlington, VA, 22202, US

(21) Appl. No.: 10/508,959

(22) PCT Filed: Apr. 3, 2003
(86) PCT No.: PCT/IL03/00279

§ 371 Date: Aug. 16, 2005

Related U.S. Application Data
(60) Provisional application No. 60/370,221, filed on Apr. 8, 2002.
(60) Provisional application No. 60/429,575, filed on Nov. 29, 2002.

(51) Int. Cl. A61K 39/00; C07K 14/47

(52) U.S. Cl. 424/185.1; 530/350

(57) ABSTRACT

A conjugate of a histone moiety covalently attached to a macromolecule-of-interest, in which the histone moiety is transportable through the cell membrane and importable into the cell nuclei, is disclosed. Further disclosed are chemical and recombinant methods of preparing such a conjugate, pharmaceutical compositions containing same and uses thereof for delivering therapeutically active macromolecules into cells. A novel method for quantitatively determining a cytoplasmic uptake and/or a nuclear uptake of a moiety into cells is also disclosed.

### FIELD AND BACKGROUND OF THE INVENTION

The present invention relates to conjugates that are capable of delivering macromolecules into cells, and, more particularly, to conjugates that comprise histone molecules covalently linked to macromolecules-of-interest. The present invention further relates to methods of preparing these conjugates, to pharmaceutical compositions containing these conjugates, and to uses thereof as delivery vehicles for delivering macromolecules into cells and in the treatment of various disorders and diseases. The present invention further relates to a method of quantitatively determine the nuclear and cytoplasmic uptake of various moieties into cells.

During the last few years, extensive efforts have been made to develop new therapeutic methods which are based on systems for delivering macromolecules, in particular nucleic acids, into animal cells. The delivery of therapeutic nucleic acids into mammalian cells is known in the art as gene therapy. Gene therapy is regarded as a potential revolution in medicine as it is aimed at eliminating the causes of diseases, whereas most of the presently used drugs treat only the symptoms. Indeed, a number of gene delivery systems, which are based on biological, chemical and physical principles, have been developed for various experimental purposes. However, gene delivery systems, as well as delivery systems of other macromolecules such as proteins, are limited by the fact that in order to be functional in the cells, the externally added macromolecules have to cross two barriers, namely the cell plasma membrane and the nuclear envelope [1]. Hence, the presently developed gene delivery systems have focused on the design of vectors that can overcome the low permeability of the cell membrane to nucleic acids and improve intracellular trafficking and nuclear delivery of genes into target cells with minimal toxicity.

The presently known gene delivery vectors are divided into three main types: viral vectors, non-viral vectors and physical vectors. Presently, many different viruses have been adapted as viral vectors, with the most advanced being retrovirus, adenovirus and adeno-associated virus. The presently adapted non-viral vectors further divide into three main categories: naked DNA, DNA complexed with cationic lipids and particles comprising condensed DNA.

Due to their high level of gene transfer efficiency, recombinant viruses are widely used as vectors for gene transfer into animal cells. It has been well established that enveloped and non-enveloped viruses are taken into intact cells via the endocytic pathway and that following their release from endosomes, their genes are translocated into the cells nuclei [2]. However, as viral vectors have fundamental problems with respect to large-scale production as well as safety issues, synthetic vehicles present several advantages over viral systems by being simple to use, easy to produce and less cytotoxic.

Over the past few years, attempts have been made to develop peptide-based gene delivery systems that can overcome both extra cellular and intracellular limitations such as cell targeting, endosome lysis and nuclear translocation, with the main goal being to identify proteins or to design short synthetic peptides that would mimic and act as efficiently as viruses for gene delivery without the limitations associated with the clinical use of viruses.

Recently, it became apparent that certain small molecular weight proteins are able to directly cross the cell plasma membrane without being susceptible to degradation by intraendosomal enzymes. The claim about direct penetration via postulated “inverted micelle pathway” [19] was based on initial observation that cellular import occurred both at 4° C. and 37° C., thus ruling out endocytosis as a possible transport mechanism [20]. A number of natural proteins or peptides, such as HIV-1 Tat, the ARM peptide derived therefrom (Tat-ARM) [21, 22] and Mastoparan, the third alpha helix from the Antennapedia homeodomain of Drosophila (penetratins) [23], have been defined as cell penetrating proteins or peptides (CPP), due to their ability to translocate cell plasma membrane independently of transporter or specific receptor. Nevertheless, the clinical use of these proteins is severely limited by the fact that these CPPs are non-human originated and therefore require manipulations of the immunological system.

Due to their ability to interact with negatively charged components present on the cell surface, as well as with DNA molecules, polycations have been also found to act as vehicles to mediate the delivery of specific genes into cells. Specifically, polylysine (PLL) [3], polyornithine (PLO) and polyethanolimine (PE1) [4] have been shown to condense DNA into small particles, which are known as polyplexes [5]. Similar to the bare polycations, polyplexes posses net positive charges and thus bind to cell surface via electrostatic interactions and are thereafter taken into the cells by the well-characterized endocytic pathway [1]. Hence, in order to reach the intranuclear space, the polyplexes should be released from the endosomal compartments and cross the nuclear envelope. Indeed, synthetic endosmolytic reagents such as chloroquine were used to improve gene transfer by the polycation-DNA complexes [6]. It has been found that chloroquine, besides blocking endosomal acidification, also promotes osmotic swelling of the endosome, which results in endosomal destabilization and the release of its content [5]. Nuclear localization signal (NLS) sequences have also been attached to the polycations or to the DNA molecules themselves in order to facilitate their nuclear entry [4, 7]. Nevertheless, the use of these polyplexes is still limited by the endosomal pathway, which unfortunately leads to extensive degradation of the genes in lysosomal compartments and/or to their poor release into the cytoplasm.

Hence, most of the presently known gene delivery systems are limited by either safety considerations (viral vectors), enhanced toxicity and/or endosomal degradation. Furthermore, most of the delivery systems that are based on proteins, polypeptides or peptide formulations with condensing and lytic peptides, are able to transfect only cultured dividing cells but not quiescent cells. The dependency on the mitotic activity of the cells is primarily because of the inability of most non-viral gene delivery systems to translocate plasmids into the nucleus of non-dividing cells.

There is thus a widely recognized need for, and it would be highly advantageous to have, a non-viral system for in vivo delivery of macromolecules (e.g., genes, proteins and other drugs) devoid of the above limitations. Such a system should utilize a delivery vehicle that would either bypass the endosomal pathway or would be translocated through the plasma membrane of non-dividing cells at neutral pH.

### SUMMARY OF THE INVENTION

The present inventors have addressed this issue by utilizing histones or peptides derived therefrom as non-viral carriers that covalently bind macromolecules, for delivery of these macromolecules into animals' cells.

Hence, according to one aspect of the present invention there is provided a conjugate comprising a histone moiety covalently linked to a macromolecule-of-interest. The histone moiety is transportable through cell membranes and importable into cell nuclei.

According to another aspect of the present invention there is provided a pharmaceutical composition comprising, as an active ingredient, the conjugate described hereinabove and a pharmaceutically acceptable carrier. The pharmaceutical composition is preferably identified for use in the treatment of a proliferative disorder or disease, a genetic disorder or disease, a bacterial infection or a viral infection.

According to yet another aspect of the present invention there is provided a method of delivering a macromolecule-of-interest into a cell. The method comprises contacting the cell with the conjugate described hereinabove. This contacting is preferably performed by co-incubating the cell and the conjugate.

According to still another aspect of the present invention there is provided a method of treating a proliferative disorder or disease, a genetic disorder or disease, a bacterial infection and/or a viral infection in a subject in need thereof. The method comprises administering to the subject a therapeutically effective amount of the conjugate described hereinabove. The macromolecule-of-interest in the conjugate has a therapeutic activity in treating the disorders or diseases delineated above.

According to an additional aspect of the present invention there is provided a polynucleotide encoding an in-frame polypeptide conjugate. The polypeptide conjugate comprises a histone moiety and a protein-of-interest, where the histone moiety is transportable through cell membranes and importable into cell nuclei.

According to further features in preferred embodiments of the invention described below, the protein-of-interest is a non-marker protein and/or has therapeutic activity.

According to yet an additional aspect of the present invention there is provided a nucleic acid construct, which comprises the polynucleotide described hereinabove. Preferably, the nucleic acid construct further comprises a cis-acting regulatory element.

According to further features in preferred embodiments of the invention described below, the histone moiety is selected from the group consisting of at least one histone protein and at least one derivative of a histone protein.

According to still further features in the described preferred embodiments the histone moiety comprises a mixture of at least two histone proteins selected from the group consisting of H1, H2A, H2B, H3 and H4. Preferably, the histone moiety comprises H2A.

According to still further features in the described preferred embodiments the macromolecule-of-interest has therapeutic activity.

According to still further features in the described preferred embodiments the macromolecule-of-interest is a non-marker macromolecule.

According to still further features in the described preferred embodiments the macromolecule-of-interest is either a chemically synthesized macromolecule or it is isolated from a biological source.

According to still further features in the described preferred embodiments the macromolecule-of-interest is a protein or a nucleic acid.

According to still further features in the described preferred embodiments the nucleic acid is an oligonucleotide, a DNA or an RNA.

According to still further features in the described preferred embodiments the nucleic acid encodes for a gene.

According to still further features in the described preferred embodiments the histone moiety is covalently linked to the macromolecule-of-interest via a spacer. Preferably, the spacer comprises a sulfide bond.

According to still further features in the described preferred embodiments the histone moiety is covalently linked to the macromolecule-of-interest via a non-peptide bond.

According to a further aspect of the present invention there is provided a method of quantitatively determining a nuclear uptake and/or a cytoplasmic uptake of a moiety into cells. The method comprises contacting the moiety with the cells; fractionating the cells into a cytoplasmic fraction and a nuclei fraction; and quantitatively determining an amount or concentration of the moiety in the cytoplasmic fraction and in the nuclei fraction.

According to further features in preferred embodiments of the invention described below, the contacting is performed by co-incubating the cells and the moiety.

According to still further features in the described preferred embodiments the fractionating is performed by permeabilizing the plasma membrane of the cells, to thereby obtain the cytoplasmic fraction and thereafter permeabilizing the nuclear membrane of the cells, to thereby obtain the nuclei fraction.

According to still further features in the described preferred embodiments the quantitatively determining comprises contacting the cytoplasmic fraction or the nuclei fraction with a solid phase having binding affinity to the moiety, to thereby adhere the moiety to the solid phase; affinity attaching a detectable molecule to the moiety; and quantitatively detecting an amount or concentration of the detectable molecule affinity bound to the moiety, to thereby quantitatively determining the amount or concentration of the moiety in the cytoplasmic fraction or in the nuclei fraction.

The solid phase is preferably selected from the group consisting of a microtiter plate, a chip and a glass. The detectable molecule preferably comprises an enzyme capable of catalyzing a colorimetric reaction, a bead, a pigment and a fluorophore.

According to still further features in the described preferred embodiments the moiety includes a detection group attached thereto. Preferably, the detection group is biotin.

According to still further features in the described preferred embodiments the moiety is a macromolecule. The macromolecule can be a protein, a nucleic acid or a histone moiety, all as described hereinabove.

Alternatively, the moiety is a conjugate of a first macromolecule covalently attached to a second macromolecule. Preferably, the first macromolecule is a histone moiety and the second macromolecule is selected from the group consisting of a protein and a nucleic acid, as described hereinabove.

The present invention successfully addresses the shortcomings of the presently known configurations by providing a conjugate of a histone moiety that is covalently linked to a macromolecule-of-interest, in which the histone moiety serves as a vehicle for transporting the macromolecule-of-interest through cell membranes and importing it into cell nuclei. Such a conjugate serves as a safe and efficient system for delivering macromolecules-of-interest into the cells, without being susceptible to endocytic degradation.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a scheme illustrating an example of the novel assay for quantitatively determining the total cellular uptake, the nuclear uptake and/or the cytoplasmic uptake of a biotinilated moiety into cells, according to a preferred embodiment of the present invention. (squares denote substrate; close circles denote biotin; stars denote horseradish peroxidase; u-shapes denote avidin and open circles denote biocytin).

FIGS. 2a-d present fluorescence micrographs showing the intracellular accumulation of a mixture of Rhodamine-labeled histone proteins in intact HeLa cells (FIGS. 2a-c) and human lymphocytes (FIG. 2d) following: 1 hour incubation of HeLa cells in the presence of a mixture of Rhodamine labeled histones (2 mM) at 37° C. (FIG. 2a), at 4° C. (FIG. 2b) and in the presence of excess unlabelled histones mixture (1:50 mole/mole) (FIG. 2c) and following 1 hour incubation of human lymphocytes in the presence of a mixture of Rhodamine labeled histones (FIG. 2d).

FIGS. 3a-g present fluorescence micrographs showing the effect of: 2 mM NaF (FIG. 3a), 20 mM Colchicine (FIG. 3b)), 5 mM Cytochalasine D (FIG. 3c), 10 mM BFA (FIG. 3d), 50 mg/ml Nystatine (FIG. 3e), 20 mM Nocadozole (FIG. 3f) and 0.5 M Sucrose (FIG. 3g) on the intracellular accumulation of a mixture of Rhodamine-labeled histones within HeLa cells (HeLa cells were incubated with the inhibitors and thereafter a 2 Mm mixture of Rhodamine-labeled histones was added for an additional 1 hour incubation at 37° C.).

FIG. 4 is a bar graph presenting the quantitative estimation of the intracellular accumulation in colon cells of externally added mixture of biotinilated histones, biotinilated BSA or biotinilated Tat-ARM, as measured by the assay of the present invention (depicted in FIG. 1), as follows: (a) Biotinilated BSA; (b) Biotinilated Tat-ARM (c) Biotinilated histones mixture incubated with colon cells at 37° C.; (d) as in (c) but incubation was performed with uncoated plates; (e) as in (c) but incubation was performed at 4° C.; (f) as in (c) but with ATP depleted cells; (g) as in (c) but with cells fixed with formaldehyde prior to the incubation period; and (h) as in (c) but in the presence of excess unlabelled histone (×100 mole/mole). The amount of biotinilated histone present in the nuclei of cells incubated at 37° C., which was 6.2 mmol histone/mg lysate, was considered as 100%. Open bars present accumulation in the nuclei; closed squares present accumulation in the cytosol.

FIG. 5 presents plots depicting kinetic studies of the penetration of histones into colon cells. Biotinilated histones (mixture) was incubated with colon cells at 37° C. in the absence (diamonds) or in the presence (squares) of 0.5 M sucrose and at 4° C. (triangles). An OD of 0.25 represents 4.7 nmol histone/mg protein.

FIGS. 6a-f present fluorescence micrographs showing the intracellular accumulation of H2A and H2B histones in HeLa cells (FIGS. 6a-d) and in human lymphocytes (FIGS. 6e-f), under the following conditions: HeLa cells were incubated for 1 hour at 37° C. in the presence of 1 mg/ml Rhodamine-labeled H2A (FIG. 6a), 1 mg/ml H2B (FIG. 6b), 1:1 (mole/mole) labeled H2A and non-labeled H2B (FIG. 6c) or 1:1 (mole/mole) labeled H2B and non-labeled H2A (FIG. 6d); Human lymphocytes were incubated for 1 hour in the presence of labeled H2A (FIG. 6e) or labeled H2B (FIG. 6f).

FIGS. 7a-d present fluorescence micrographs showing the intracellular accumulation of H3 and H4 following incubation of HeLa cells for 1 hour at 37° C. in the presence of: 1 mg/ml Rhodamine-labeled H3 (FIG. 7a), and 1 mg/ml Rhodamine-labeled H4 (FIG. 7b), 1:1 (mole/mole) Rhodamine-labeled H3 and non-labeled H4 (FIG. 7c) or 1:1 (mole/mole) Rhodamine-labeled H4 and non-labeled H3 (FIG. 7d).

FIG. 8a is a bar graph presenting the quantitative estimation of the penetration of biotinilated BSA, biotinilated histones mixture and biotinilated pure histones into intact HeLa cells, as follows: (a) Biotinilated BSA (1 mg/ml); (b) biotinilated histones mixture (c) biotinilated H2A; (d) biotinilated H2B; (e) as in (d) but in the presence of unlabelled H2A (1:1 mole/mole); (f) biotinilated H3; (g) as in (f) but in the presence of unlabelled H4 (1:1 mole/mole); (h) biotinilated H4 (0.1 mg/ml); (i) as in (h) but in the presence of unlabelled H3 (1:1 mole/mole). An O.D. of 0.2 represents 0.47 nmol histone/mg protein.

FIG. 8b is a bar graph presenting the quantitative estimation of the penetration of biotinilated H2A into colon cells, as follows: (a) Biotinilated BSA; (b) Biotinilated H2A incubated with colon cells at 37° C.; (c) as in (b) but with ATP depleted cells; (d) as in (b) but with cells fixed with formaldehyde prior to the incubation period; (e) as in (b) but incubation was performed with uncoated plates; (f) as in (c) but in the presence of excess unlabelled H2B (×60 mole/mole). The amount of biotinilated H2A present in the nuclei of cells incubated in 37° C., 5.8 nmol/mg protein, was considered as 100%. Open bars present accumulation in the nuclei; closed bars present accumulation in the cytosol.

FIGS. 9a-e present fluorescence micrographs (FIGS. 9a-c) and confocal micrographs (FIGS. 9d-e) showing the intracellular accumulation of Rhodamine-labeled BSA-histone conjugates in intact HeLa cells, following 1 hour incubation of HeLa cells in the presence of: Rhodamine-labeled BSA (FIG. 9a), labeled BSA-histone conjugate (FIGS. 9b and 9d), labeled BSA-histone conjugate in the presence of excess unlabeled histones mixture (1:50 mole/mole) (FIG. 9c) and labeled BSA-H2A (FIG. 9e).

FIG. 10 is a bar graph presenting quantitative estimation of the accumulation of externally added biotinilated BSA-histone conjugates in colon cells cytosol, as follows: (a) Biotinilated BSA; (b) biotinilated BSA conjugated to a peptide bearing the NLS of the large T antigen of the SV40; (c) Biotinilated BSA-histones mixture conjugates incubated with colon cells at 37° C.; (d) as in (c) but at 4° C. (e) as in (c) but incubation was performed with uncoated plates; (f) as in (c) but with ATP depleted cells; (g) as in (c) but with cells fixed with formaldehyde prior to the incubation period; (h) as in (c) but in the presence of a mixture of unlabelled histones (1:1 mole/mole); (i) as in (h) but in the presence of excess unlabelled histones (×50 mole/mole); (j) as in (h) but with prefixed cells. The amount of biotinilated BSA-histone conjugate present in the nuclei of cells incubated at 37° C., which was 6.3 nmol/mg protein, was considered as 100%. Open bars present accumulation in the nuclei; closed bars present accumulation in the cytosol.

FIG. 11a is a bar graph presenting the quantitative estimation of the cellular and nuclear uptake of biotinilated BSA-H2A conjugates in colon cells, as follows: (a) Biotinilated BSA; (b) Biotinilated BSA-H2A conjugates incubated with colon cells at 37° C.; (c) as in (b) but with cells fixed with formaldehyde prior to the incubation period; (d) as in (b) but with ATP depleted cells; (e) as in (b) but incubation was performed with uncoated plates; (f) as in (b) but in the presence of unlabelled H2B (1:1 mole/mole). The amount of biotinilated BSA-H2A present in the nuclei of cells incubated in 37° C., which was 6.4 nmol/mg protein, was considered as 100%. Open bars present accumulation in the nuclei; closed bars present accumulation in the cytosol.

FIG. 11b is a bar graph presenting the quantitative estimation of the cellular and nuclear uptake of biotinilated BSA-H2B conjugates in colon cells, as follows: (a) Bb-Biotinilated BSA; (b) Biotinilated BSA-H2B conjugates incubated with colon cells at 37° C.; (c) as in (b) but in the presence of unlabelled H2A (1:1 mole/mole); (d) as in (c) but in the presence of excess unlabelled H2A (1:2) mole/mole); (e) as in (c) but in the presence of excess unlabelled H2A (1:3) mole/mole); (f) as in (c) but with cells fixed with formaldehyde prior to the incubation period; (g) as in (c) but incubation was performed with uncoated plates; (h) as in (c) but with ATP depleted cells; (i) as in (c) but incubation was performed at 4° C. The amount of biotinilated BSA-H2B present in the nuclei of cells incubated in 37° C., which was 6.2 nmol/mg protein, was considered as 100%. Open bars present accumulation in the nuclei; closed bars present accumulation in the cytosol.

FIG. 12 presents comparative plots demonstrating the penetration of biotinilated BSA-Tat-ARM conjugate and the biotinilated BSA-histone conjugate of the present invention into intact colon cells. The biotinilated conjugates at increasing concentrations were incubated with intact Colon cells for 1 hour at 37° C. and the amount of biotinilated molecules within the cell lysate was estimated by the quantitative assay of the present invention. Squares denote biotinilated BSA-histone conjugate; triangles denote biotinilated BSA-Tat-ARM conjugate. An O.D. of 0.50 represents 9.0 nmol histone/mg protein.

### DESCRIPTION OF THE PREFERRED EMBODIMENTS

The present invention is of (i) novel conjugates that comprise histone moieties covalently linked to macromolecules-of-interest; (ii) pharmaceutical compositions containing same; (iii) methods of preparing same; (vi) uses thereof as delivery vehicles for delivering macromolecules-of-interest into cells; and (v) uses thereof in the treatment of disorders or diseases such as, but not limited to, proliferative disorders and diseases, genetic disorders and diseases, bacterial infections and viral infections. The present invention is further of polynucleotides encoding in-frame polypeptide conjugates (i.e., chimeric polypeptides) that comprise histone moieties covalently linked to a protein-of-interest, of nucleic acid constructs containing same, and of a novel method for quantitatively determining the cellular uptake of a moiety into cells.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Histones are small, positively charged polypeptides that are rich in basic amino acids. Hence, histone molecules share several properties with basic macromolecules such as the polycations PLL, PLO and PEI described in the Background section [8]. However, as opposed to these synthetic polycations, histone molecules also bear well conserved NLS [9] sequences and are thus readily imported into cells nuclei.

Histone proteins are synthesized in the cytoplasm however in order to function as the nucleosomal core they must be transported post translationally to the nucleoplasm. There are five classes of histones, termed H1, H2A, H2B, H3, and H4, which associate to form a disk-shaped octomeric protein core. In eukaryotic cells, genomic DNA associates with histones, as well as with other proteins, to form a compact complex called chromatin. The DNA winds around the protein core, such that the basic, positively charged, amino acids of the histones interact with the negatively charged phosphate groups of the DNA. Approximately 146 base pairs of DNA wrap around a histone core to make up a nucleosomal core [10].

The histone octamers are able to form complexes with DNA molecules also under in vitro conditions [8]. Histone octamer-DNA complexes or other complexes formed between DNA molecules and isolated histone (such as H2A) similar to the polyplexes, have been used to transfect animal cells [11, 12, 13]. Complexes formed between H1 and DNA molecules were similarly used. Interestingly, it was found that galactosylation of histone H1 led to the formation of targeted complexes that specifically transfect cells expressing the asialoglycoprotein receptor [14]. Recently it has been shown that a peptide derived from the histone H2A was able to electrostatically bind DNA molecules [15]. Polyplexes formed between the H2A derived peptide and DNA molecules were able to mediate transfer of a plasmid encoding the beta-galactosidase gene into COS-7 cells.

In spite of the extensive use of histone-DNA polyplexes for transfection of animal cells, very few studies have been conducted to elucidate the mechanism by which such complexes are taken up by animal cells [16]. It was generally assumed that as in the case of certain virus particles, polycations-DNA polyplexes and histone-DNA polyplexes are internalized into the cell via clatherin coated pits [17]. Hence, it was considered that the penetration of histone molecules to cells is mediated via endocytic pathway [18] and is therefore affected by endosomal degradation.

While evaluating and studying the mechanism by which histone molecules penetrate the cell, the present inventors have surprisingly found that histone molecules are able to directly penetrate the plasma membrane of cells and even accumulate within the nucleoplasm, in a non-endocytic pathway. These studies have led the present inventors to develop a delivery system in which macromolecules-of-interest are covalently bound to histone molecules. Such systems are superior to the presently known histone-DNA non-covalent polyplexes since they enable intracellular delivery of macromolecules-of-interest, such as therapeutic macromolecules, which do not naturally complex with histones (e.g., RNA, proteins and the like).

Thus, according to one aspect of the present invention, there is provided a novel conjugate which enables intracellular and intranuclear delivery of macromolecules-of-interest.

The conjugate of the present invention includes a histone moiety covalently linked to a macromolecule-of-interest. As is further described hereinabove and in the Examples section which follows, the histone moiety of the conjugate is capable of transporting through cell membranes and importing into cell nuclei, and therefore enables the translocation of the macromolecule-of interest covalently attached thereto into the cell cytoplasm and nucleoplasm.

To insure plasma membrane permeability and nuclear transport, the histone moiety utilized in the conjugate of the present invention includes two structural elements; a positively charged amino acid sequence and a nuclear localization signal (NLS).

The histone moiety utilized by the present invention, can be for example, at least a portion of a histone such as the H1 histone protein (GenBank Accession No. AF 531304), H2A histone protein (GenBank Accession No. M 60752), H2B histone protein (GenBank Accession No. M 60751), H3 histone protein (GenBank Accession No. M 26150), and H4 histone protein (GenBank Accession No. M 60749) or any combination of two or more histone proteins either covalently linked therebetween, provided as a mixture or fused in frame (chimera). In a preferred embodiment, the histone moiety includes a mixture of all the five histones described hereinabove. This mixture is termed herein, interchangeably, as “histones mixture” or “a mixture of histones”.

In cases where the conjugate of the present invention utilizes a single histone moiety the H2A histone protein or a portion thereof is preferably utilized. As is demonstrated in the Examples section that follows, the H2A histone exhibits the highest penetration activity into cells, and in particular, into cell nuclei.

It will be appreciated that the histone moiety of the conjugate of the present invention can also be a modified histone protein or proteins, or a derivative of such proteins.

A derivative of a histone protein can be a natural or synthetic peptide or polypeptide that includes a sequence derived from a histone protein. A modified histone includes a histone sequence that is at least in part modified (e.g., incorporates non-natural amino acids).

Preferably, the derivatives of the histone proteins utilized by the conjugates of the present invention include one or more modifications that enhance cellular and/or nuclear penetration (e.g., by presenting more positively charged residues).

In one embodiment, the histone moiety comprises an active portion of a histone protein with or without further modifications that includes a sufficient number of positively charged amino acid residues so as to facilitate endocytosis-free crossing of cell membranes and an NLS sequence for importing into cell nuclei.

The histone moiety, according to the present invention, can include either a single derivative of a histone protein or a combination of two or more derivatives of histone proteins. Similarly, the histone moiety can include a combination of one or more derivatives of histone proteins and one or more histone proteins.

As described hereinabove the histone moiety of the present invention is covalently linked to a macromolecule-of-interest to form the conjugate of the present invention.

As used herein the phrase “macromolecule-of-interest” refers to a nucleic acid (i.e., polynucleotide), a protein (i.e., polypeptide), or any other molecule which can be chemically synthesized or isolated from a natural source. Preferably, the macromolecule of the present invention has therapeutic activity while being a non-marker macromolecule.

The term “nucleic acid” refers to a single stranded or double stranded, oligomer (i.e., oligonucleotide) or polymer (i.e., polynucleotide), of ribonucleic acid (RNA) or deoxyribonucleic acid (DNA) or mimetics hereof. These terms include oligonucleotides and/or polynucleotides composed of naturally occurring bases, sugars and covalent internucleoside linkages (e.g., backbone) as well as nucleic acid sequences having non-naturally-occurring portions which function similarly.

The polynucleotides of the present can be used for intracellular expression of an RNA or polypeptide product. This is of special significance in cases of severe aberrancies in gene expression. For example multiple mutations in transforming growth factor (TGF)-beta have been associated with high occurrence of cleft palate in both mice and humans. Apparently, TGF-beta is required for the adhesion and intercalation of medial edge epithelial cells during palate fusion [Tudela (2002) Int. J. Dev. Biol. 46(3):333-6]. Therefore efficient expression of wild-type. TGF-beta polynucleotide using the conjugates of the present invention is of importance in the prevention of disorders such as cleft palate even in embryonal stages of development.

The polynucleotide of the present invention can be isolated from a natural source and provided in the form of an RNA sequence, a complementary polynucleotide sequence (cDNA), a genomic polynucleotide sequence and/or a composite polynucleotide sequences (e.g., a combination of the above).

As used herein the phrase “complementary polynucleotide sequence” refers to a sequence, which results from reverse transcription of messenger RNA using a reverse transcriptase or any other RNA dependent DNA polymerase. Such a sequence can be subsequently amplified in vivo or in vitro using a DNA dependent DNA polymerase.

As used herein the phrase “genomic polynucleotide sequence” refers to a sequence derived (isolated) from a chromosome and thus it represents a contiguous portion of a chromosome.

As used herein the phrase “composite polynucleotide sequence” refers to a sequence, which is at least partially complementary and at least partially genomic. A composite sequence can include some exonal sequences required to encode the polypeptide of the present invention, as well as some intronic sequences interposing therebetween. The intronic sequences can be of any source, including of other genes, and typically will include conserved splicing signal sequences. Such intronic sequences may further include cis acting expression regulatory elements.

A variety of techniques for extracting nucleic acids from biological samples are known in the art. For example, see those described in Maniatis et al., Molecular Cloning: A Laboratory Manual (New York, Cold Spring Harbor Laboratory, 1982); Arrand, Preparation of Nucleic Acid Probes, in pp. 18-30, Nucleic Acid Hybridization: A Practical Approach (Ed Hames and Higgins, IRL Press, 1985); or, in PCR Protocols, Chapters 18-20 (Innis et al., ed., Academic Press, 1990).

The nucleic acid conjugated to the histone moiety of the present invention can also be an oligonucleotide.

Preferably, oligonucleotides used according to the present invention are those having a length selected from a range of 10 to about 800 bases.

Oligonucleotides can be generated according to any oligonucleotide synthesis method known in the art such as enzymatic synthesis or solid phase chemical synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art.

The oligonucleotides of the present invention may comprise heterocylic nucleosides consisting of purines and the pyrimidines bases, bonded in a 3′ to 5′ phosphodiester linkage.

Preferably used oligonucleotides are those modified in either backbone, internucleoside linkages or bases, as is broadly described hereinunder. Such modifications can oftentimes facilitate oligonucleotide uptake and resistivity to intracellular conditions.

Specific examples of preferred oligonucleotides useful according to the present invention include oligonucleotides containing modified backbones or non-natural internucleoside linkages. Oligonucleotides having modified backbones include those that retain a phosphorus atom in the backbone, as disclosed in U.S. Pat. Nos. 687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Preferred modified oligonucleotide backbones include, for example, phosphorothioates, chiral phosphorothioates, phosphorodithioates, phosphotriesters, aminoalkyl, phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms can also be used.

Alternatively, modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

Other oligonucleotides which can be used according to the present invention, are those modified in both sugar and the internucleoside linkage, i.e., the backbone, of the nucleotide units are replaced with novel groups. The base units are maintained for complementation with the appropriate polynucleotide target. An example for such an oligonucleotide mimetic includes peptide nucleic acid (PNA). A PNA oligonucleotide refers to an oligonucleotide where the sugar-backbone is replaced with an amide containing backbone, in particular an aminoethylglycine backbone. The bases are retained and are bound directly or indirectly to aza nitrogen atoms of the amide portion of the backbone. U.S. patents that teach the preparation of PNA compounds include, but are not limited to, U.S. Pat. Nos. 5,539,082; 5,714,331; and 5,719,262, each of which is herein incorporated by reference. Other backbone modifications, which can be used in the present invention, are disclosed in U.S. Pat. No. 6,303,374.

Oligonucleotides of the present invention may also include base modifications or substitutions. As used herein, “unmodified” or “natural” bases include the purine bases adenine (A) and guanine (G), and the pyrimidine bases thymine (T), cytosine (C) and uracil (U). Modified bases include but are not limited to other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further bases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Such bases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. [Sanghvi Y S et al. (1993) Antisense Research and Applications, CRC Press, Boca Raton 276-278] and are presently preferred base substitutions, even more particularly when combined with 2′-O-methoxyethyl sugar modifications.

It is not necessary for all positions in a given oligonucleotide molecule to be uniformly modified, and in fact more than one of the aforementioned modifications may be incorporated in a single compound or even at a single nucleoside within an oligonucleotide.

The oligonucleotide of the conjugate of the present invention can encode for an active domain of a highly expressed polypeptide of interest (e.g., oncogenes), thereby generating, for example, a dominant-negative effect upon sequestration of endogenous effectors. For example the oligonucleotide of the present invention can encode the extracellular domain of the epidermal growth factor receptor (EGFR). It is recognized that overexpression of the latter is associated with high-grade astrocytomas that affect adults, such as glioblastoma multiforme [Louis (1994) Baillieres Clin. Neurol. 3(2):335-52]. Therefore, sequestration of EGF by ectopic expression of the EGFR-ligand binding domain can reduce endogenous receptor activity and hence its therapeutic value.

The oligonucleotide can also be configured for down-regulating expression of a gene or suppression of a gene product activity.

Selective down-regulation of gene expression is desired in many cases where genetic over-expression is associated with disease progression. For example, it is recognized that over expression of ErbB-2 is associated with poor prognosis of breast cancer patients [Forseen (2002) Anticancer Res. 22:1599-602].

However, currently available genetic tools (i.e., antisense molecules, small interfering double stranded RNA (siRNA) etc.), for inhibiting gene expression are mostly limited by poor cellular uptake.

Thus, the conjugates of the present invention can include antisense or siRNA oligonucleotides selected capable of efficiently suppressing gene expression.

The antisense oligonucleotides of the conjugates preferably contain two or more chemically distinct regions, each made up of at least one nucleotide. These oligonucleotides typically contain at least one region wherein the oligonucleotide is modified so as to confer upon the oligonucleotide increased resistance to nuclease degradation, increased cellular uptake, and/or increased binding affinity for the target polynucleotide. An additional region of the oligonucleotide may serve as a substrate for enzymes capable of cleaving RNA:DNA or RNA:RNA hybrids. An example for such includes RNase H, which is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

The antisense oligonucleotides utilized by the present invention may be formed as composite structures of two or more oligonucleotides, or modified oligonucleotides, as described above. Representative U.S. patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein fully incorporated by reference.

The oligonucleotides utilized by the present invention can also include a ribozyme sequence. Ribozymes are being increasingly used for the sequence-specific inhibition of gene expression by the cleavage of mRNAs. Several ribozyme sequences can be fused to the oligonucleotides of the present invention. These sequences include but are not limited ANGIOZYME specifically inhibiting formation of the VEGF-R (Vascular Endothelial Growth Factor receptor), a key component in the angiogenesis pathway, and HEPTAZYME, a ribozyme designed to selectively destroy Hepatitis C Virus (HCV) RNA, (Ribozyme Pharmaceuticals, Incorporated—WEB home page www.rpi.com).

As mentioned hereinabove, the conjugates of the present invention can also include small interfering duplex oligonucleotides [i.e., small interfering RNA (siRNA)], which direct sequence specific degradation of mRNA through the previously described mechanism of RNA interference (RNAi) [Hutvagner and Zamore (2002) Curr. Opin. Genetics and Development 12:225-232].

As used herein, the phrase “duplex oligonucleotide” refers to an oligonucleotide structure or mimetics thereof, which is formed by either a single self-complementary nucleic acid strand or by at least two complementary nucleic acid strands. The “duplex oligonucleotide” of the present invention can be composed of double-stranded RNA (dsRNA), a DNA-RNA hybrid, single-stranded RNA (ssRNA), isolated RNA (i.e., partially purified RNA, essentially pure RNA), synthetic RNA and recombinantly produced RNA.

Instructions for generation of duplex oligonucleotides capable of mediating RNA interference are provided in www.ambion.com.

As described hereinabove, the macromolecule-of-interest of the present invention can also be a protein. The term “protein”, which is also referred to herein interchangeably as “polypeptide”, refers to an amino acid sequence of any length including full-length proteins or portions thereof, wherein the amino acid residues are linked by covalent peptide bonds.

The term “peptide” as used herein encompasses native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells. Such modifications include, but are not limited to N-terminus modification, C-terminus modification, peptide bond modification, including, but not limited to, CH₂—NH, CH₂—S, CH₂—S═O, O═C—NH, CH₂—O, CH₂—CH₂, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated bonds (—N(CH₃)—CO—), ester bonds (—C(R)H—C—O—O—C(R)—N—), ketomethylene bonds (—CO—CH₂—), α-aza bonds (—NH—N(R)—CO—), wherein R is any alkyl, e.g., methyl, carbamine bonds (—CH₂—NH—), hydroxyethylene bonds (—CH(OH)—CH2—), thioamide bonds (—CS—NH—), olefinic double bonds (—CH═CH—), retro amide bonds (—NH—CO—), peptide derivatives (—N(R)—CH₂—CO—), wherein R is the “normal” side chain, naturally presented on the carbon atom.

These modifications can occur at any of the bonds along the peptide chain and even at several (2-3) at the same time.

Natural aromatic amino acids, Trp, Tyr and Phe, may be substituted for synthetic non-natural acid such as Phenylglycine, TIC, naphthylelanine (Nol), ring-methylated derivatives of Phe, halogenated derivatives of Phe or o-methyl-Tyr.

In addition to the above, the peptides of the present invention may also include one or more modified amino acids or one or more non-amino acid monomers (e.g. fatty acids, complex carbohydrates etc).

As used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Tables 1 and 2 below list naturally occurring amino acids (Table 1) and non-conventional or modified amino acids (Table 2) which can be used with the present invention.

TABLE 1

| | Three-Letter | |
|---|---|---|
| Amino Acid | Abbreviation | One-letter Symbol |
| alanine | Ala | A |
| Arginine | Arg | R |
| Asparagine | Asn | N |
| Aspartic acid | Asp | D |
| Gysteine | Cys | C |
| Clutamine | Gln | Q |
| Glutamic Acid | Glu | E |
| glycine | Gly | G |
| Histidine | His | H |
| isoleucine | Iie | I |
| leucine | Leu | L |
| Lysine | Lys | K |
| Methionine | Met | M |
| phenylalanine | Phe | F |
| Proline | Pro | P |
| Serine | Ser | S |
| Threonine | Thr | T |
| tryptophan | Trp | W |
| tyrosine | Tyr | Y |
| Valine | Val | V |
| Any amino acid as above | Xaa | X |

TABLE 2

| Non-conventional amino acid | Code | Non-conventional amino acid | Code |
|---|---|---|---|
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgin |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcyclopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cyclododeclglycine | Ncdod |
| D-α-methylalnine | Dnmala | N-cyclooctylglycine | Ncoct |
| D-α-methylarginine | Dnmarg | N-cyclopropylglycine | Ncpro |
| D-α-methylasparagine | Dnmasn | N-cycloundecylglycine | Ncund |
| D-α-methylasparatate | Dnmasp | N-(2,2-diphenylethyl)glycine | Nbhm |
| D-α-methylcysteine | Dnmcys | N-(3,3-diphenylpropyl)glycine | Nbhe |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)gycine | Nile | D-N-methylserine | Dnmser |
| N-(2-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nva |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomo phenylalanine | Mhphe |
| L-α-methylisoleucine | Mile | N-(2-methylthioethyl)glycine | Nmet |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylbistidine | Dnmhis | N-(hydroxyethyl)glycine | Nser |
| D-N-methylisoleucine | Dnmile | N-(imidazolylethyl)glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmetbionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | mser | L-α-methylthreonine | Mthr |
| L-α-methylvaline | Mtrp | L-α-methyltyrosine | Mtyr |
| L-α-methylleucine | Mval Nnbhm | L-N-methylhomophenylalanine | Nmhphe |
| N-(N-(2,2-diphenylethyl) | Nnbhm | N-(N-(3,3-diphenylpropyl) | Nnbhe |
| carbamylmethyl-glycine | Nmbc | carbamylmethyl(1)glycine | |
| 1-carboxy-1-(2,2-diphenyl | | | |
| ethylamino)cyclopropane | | | |

When utilized in therapeutics, the polypeptide of the conjugate is preferably provided in soluble form. In such cases, the polypeptide preferably includes one or more non-natural or natural polar amino acids, including but not limited to serine and threonine, which are capable of increasing polypeptide solubility due to their hydroxyl-containing side chain.

The peptides of the conjugates of the present invention are preferably utilized in a linear form, although it will be appreciated that in cases where cyclization does not severely interfere with peptide characteristics, cyclic forms of the peptide can also be utilized.

Cyclic peptides can either be synthesized in a cyclic form or configured so as to assume a cyclic form under desired conditions (e.g., physiological conditions).

For example, a peptide according to the teachings of the present invention can include at least two cysteine residues flanking the core peptide sequence. In this case, cyclization can be generated via formation of S—S bonds between the two Cys residues. Side-chain to side chain cyclization can also be generated via formation of an interaction bond of the formula —(—CH2—)n-S—CH₂—C—, wherein n=1 or 2, which is possible, for example, through incorporation of Cys or homoCys and reaction of its free SH group with, e.g., bromoacetylated Lys, Orn, Dab or Dap. Furthermore, cyclization can be obtained, for example, through amide bond formation, e.g., by incorporating Glu, Asp, Lys, Orn, di-amino butyric (Dab) acid, di-aminopropionic (Dap) acid at various positions in the chain (—CO—NH or —NH—CO bonds). Backbone to backbone cyclization can also be obtained through incorporation of modified amino acids of the formulas H—N((CH₂)n-COOH)—C(R)H—COOH or H—N((CH₂)n-COOH)—C(R)H—NH₂, wherein n=1-4, and further wherein R is any natural or non-natural side chain of an amino acid.

The peptides of the conjugates of the present invention can be chemically synthesized. Synthetic peptides can be prepared by classical methods known in the art, for example, by using standard solid phase techniques. The standard methods include exclusive solid phase synthesis, partial solid phase synthesis methods, fragment condensation, classical solution synthesis, and even by recombinant DNA technology. See, e.g., Merrifield, J. Am. Chem. Soc., 85:2149 (1963), incorporated herein by reference. Solid phase peptide synthesis procedures are well known in the art and further described by John Morrow Stewart and Janis Dillaha Young, Solid Phase Peptide Syntheses (2nd Ed., Pierce Chemical Company, 1984).

Synthetic peptides can be purified by preparative high performance liquid chromatography [Creighton T. (1983) Proteins, structures and molecular principles. WH Freeman and Co. N.Y.] and the composition of which can be confirmed via amino acid sequencing.

Alternatively, the polypeptides of the present invention can be isolated from a biological source (e.g., a biological sample).

The phrase “biological sample” includes any body sample such as blood (serum or plasma), sputum, ascites fluids, pleural effusions, urine, biopsy specimens, isolated cells and/or cell membrane preparation. Peptides isolated from biological samples can be naturally occurring peptides or degradation products of polypeptides or proteins.

Protein purification methods are well known in the art. Examples include but are not limited to fractionation of samples by ammonium sulfate precipitation and acid or chaotrope extraction. Exemplary purification steps may include hydroxyapatite, size exclusion, FPLC and reverse-phase high performance liquid chromatography. Suitable chromatographic media include derivatized dextrans, agarose, cellulose, polyacrylamide, specialty silicas, and the like. PEI, DEAE, QAE and Q derivatives are preferred. Exemplary chromatographic media include those media derivatized with phenyl, butyl, or octyl groups, such as Phenyl-Sepharose FF (Pharmacia), Toyopearl butyl 650 (Toso Haas, Montgomeryville, Pa.), Octyl-Sepharose (Pharmacia) and the like; or polyacrylic resins, such as Amberchrom CG 71 (Toso Haas) and the like. Suitable solid supports include glass beads, silica-based resins, cellulosic resins, agarose beads, cross-linked agarose beads, polystyrene beads, cross-linked polyacrylamide resins and the like that are insoluble under the conditions in which they are to be used. These supports may be modified with reactive groups that allow attachment of proteins by amino groups, carboxyl groups, sulfhydryl groups, hydroxyl groups and/or carbohydrate moieties. Examples of coupling chemistries include cyanogen bromide activation, N-hydroxysuccinimide activation, epoxide activation, sulfhydryl activation, hydrazide activation, and carboxyl and amino derivatives for carbodiimide coupling chemistries. These and other solid media are well known and widely used in the art, and are available from commercial suppliers. Selection of a particular method is preferably determined by the properties of the chosen support. See, for example, Affinity Chromatography: Principles & Methods, Pharmacia LKB Biotechnology, Uppsala, Sweden, 1988.

The polypeptides of the present invention can be isolated by exploitation of their biochemical, structural, and biological properties. For example, immobilized metal ion adsorption (IMAC) chromatography can be used to purify histidine-rich proteins, including those comprising polyhistidine tags. Briefly, a gel is first charged with divalent metal ions to form a chelate (Sulkowski, Trends in Biochem. 3:1-7, 1985). Histidine-rich proteins will be adsorbed to this matrix with differing affinities, depending upon the metal ion used, and will be eluted by competitive elution, lowering the pH, or use of strong chelating agents. Other methods of purification include purification of glycosylated proteins by lectin affinity chromatography and ion exchange chromatography (Methods in Enzymol., Vol. 182, “Guide to Protein Purification”, M. Deutscher, (ed.), Acad. Press, San Diego, 1990, pp. 529-39).

As described hereinabove, the histone moiety and the macromolecule-of-interest of the present invention are covalently linked, through, for example, a peptide bond.

In cases where direct peptide bonding of the conjugate of the present invention is limited by (i) steric hindrance of the histone moiety; (ii) elevated susceptibility to intracellular proteases; and/or (iii) inviolability of amine groups for peptide bonding in most macromolecules (e.g., purines), the two moieties of the conjugates of the present invention can be linked via, for example, a spacer.

The spacer can be, for example, a chain that includes 2-20 carbon atoms, preferably 2-15 carbon atoms and, most preferably, 2-10 carbon atoms. The chain can be saturated or unsaturated.

The chain can be interrupted by one or more heteroatoms such as, but not limited to, O, S and N. The chain can be further substituted by one or more chemical groups such as, but not limited to, ═O, ═NH and an alkyl group having 1-3 carbon atoms. The chain can include or can be substituted by a cyclic group such as, but not limited to, saturated or unsaturated cycloalkyl, aryl, heteroaryl, and heteroalicyclic group.

Preferably, the spacer is linked at one end to one or more free amino groups of the histone moiety or the macromolecule. The other end of the spacer preferably includes a group that is amenable to electrophilic or nucleophilic attack by a free functional group of the other moiety. Such a free functional group can be, for example, an amino group, a hydroxyl group and a thiol group. Examples groups amenable to electrophilic or nucleophilic attack include, without limitation, an unsaturated group such as an aryl, an unsaturated cycloalkyl and an unsaturated cycloalkyl substituted by an electron-withdrawing group.

In a preferred embodiment of the present invention, the functional group is a thiol group and hence the spacer includes a sulfide bond.

Any of the conjugate of the present invention described herein can be produced either chemically or via well known recombinant approaches.

Thus, according to another aspect of the present invention, there is provided a method of synthesizing the conjugates of the present invention.

According to one embodiment of the present invention, the method is effected by covalently linking the histone moiety and the macromolecule-of-interest.

The histone moiety and the macromolecule can be linked directly via a peptide bond, or via a spacer, as is described in detail hereinabove.

The covalent linking of the two moieties can be performed, for example, by first obtaining a histone moiety, either recombinantly or from a commercial source, purifying the moiety, if necessary, and then chemically reacting the histone with the selected macromolecule, using techniques known to those skilled in the art.

Preferably, the moieties are linked via a spacer (described above) and the synthesis involves introduction of a spacer to one moiety and a functional group to the other moiety, such that the covalent linking is performed between the spacer and the functional group.

More specifically, is cases where the synthesis involves a spacer, the method, according to this aspect of the present invention, is preferably effected by first attaching a spacer to either the histone moiety or the macromolecule. In order to facilitate the reaction between the spacer and the second moiety, the later is preferably converted into a functionalized derivative thereof, which comprises a free functional group.

The phrase “a functional group” describes a chemically reactive group such as, but not limited to, amine, hydroxyl, thiol, halide and acyl halide. The functional group is selected so as to easily react with the spacer by any known chemical reaction. However, preferred reactions include simple nucleophilic, nucleophilic-addition or electrophilic reactions. Hence, the spacer that is attached to the first moiety preferably includes a group that can react with the functional group of the second moiety via such reactions.

According to a preferred embodiment of the present invention, the functional group is a thiol group and the functionalized derivative is a thiolated derivative.

As is described hereinabove, the spacer preferably includes a group that is susceptible to electrophilic or nucleophilic attack by a free functional group of the other moiety. Examples to such a group that is easily reacted with the thiolated derivative described hereinabove include, without limitation, an unsaturated group such as an aryl, an unsaturated cycloalkyl and an unsaturated cycloalkyl substituted by an electron-withdrawing group.

An exemplary method of preparing conjugates via thiol functional group is disclosed in Theodore et al, J. Neurosci. (1995) 15(11):7158.

In cases where large amounts of the conjugates of the present invention are desired and provided that the macromolecule-of interest is a polypeptide, the conjugates of the present invention can be generated using recombinant techniques.

Thus, according to another embodiment of this aspect of the present invention, an expression construct (i.e., expression vector), which is also referred to herein interchangeably as “a nucleic acid construct”, which includes a polynucleotide encoding the polypeptide conjugate of the present invention (i.e., a chimera including the histone moiety and the polypeptide macromolecule-of-interest) positioned under the transcriptional control of a regulatory element, such as a promoter, is introduced into host cells.

The “transformed” cells are cultured under suitable conditions, which allow the expression of the fusion protein encoded by the polynucleotide.

Following a predetermined time period, the expressed fusion protein is recovered from the cell or cell culture, and purification is effected according to the end use of the recombinant polypeptide.

Depending on the host/vector system utilized, any of a number of suitable transcription and translation elements including constitutive and inducible promoters, transcription enhancer elements, transcription terminators, and the like, can be used in the expression vector [see, e.g., Bitter et al., (1987) Methods in Enzymol. 153:516-544].

Other then containing the necessary elements for the transcription and translation of the inserted coding sequence (encoding the chimera), the expression construct of the present invention can also include sequences engineered to optimize stability, production, purification, yield or toxicity of the expressed fusion protein.

For example, a cleavable fusion protein can be engineered to include the conjugate of the present invention and a cleavable moiety. Such a fusion protein can be designed so that the fusion protein can be readily isolated by affinity chromatography; e.g., by immobilization on a column specific for the cleavable moiety. Where a cleavage site is engineered between the conjugate and the cleavable moiety, the conjugate can be released from the chromatographic column by treatment with an appropriate enzyme or agent that disrupts the cleavage site [e.g., see Booth et al. (1988) Immunol. Lett. 19:65-70; and Gardella et al., (1990) J. Biol. Chem. 265:15854-15859].

A variety of prokaryotic or eukaryotic cells can be used as host-expression systems to express the fusion protein coding sequence. These include, but are not limited to, microorganisms, such as bacteria transformed with a recombinant bacteriophage DNA, plasmid DNA or cosmid DNA expression vector containing the conjugate coding sequence; yeast transformed with recombinant yeast expression vectors containing the conjugate coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the conjugate coding sequence. Mammalian expression systems can also be used to express the conjugate of the present invention. Bacterial systems are preferably used to produce recombinant proteins since they enable a high production volume at low cost.

In bacterial systems, a number of expression vectors can be advantageously selected depending upon the use intended for the conjugate expressed. For example, when large quantities of conjugates are desired, vectors that direct the expression of high levels of the protein product, possibly as a fusion with a hydrophobic signal sequence, which directs the expressed product into the periplasm of the bacteria or the culture medium where the protein product is readily purified may be desired. Certain fusion protein engineered with a specific cleavage site to aid in recovery of the conjugate may also be desirable. Such vectors adaptable to such manipulation include, but are not limited to, the pET series of E. coli expression vectors [Studier et al. (1990) Methods in Enzymol. 185:60-89).

It will be appreciated that when codon usage for a human, plant or yeast gene is inappropriate for expression in E. coli, the host cells can be co-transformed with vectors that encode species of tRNA that are rare in E. coli but are frequently used by in other organisms. For example, co-transfection of the gene dnaY, encoding tRNA. ArgAGA/AGG, a rare species of tRNA in E. coli, can lead to high-level expression of heterologous plant genes in E. coli. [Brinkmann et al., Gene 85:109 (1989) and Kane, Curr. Opin. Biotechnol. 6:494 (1995)]. The dnaY gene can also be incorporated in the expression construct such as for example in the case of the pUBS vector (U.S. Pat. No. 6,270,0988).

In yeast, a number of vectors containing constitutive or inducible promoters can be used, as disclosed in U.S. Pat. No. 5,932,447. Alternatively, vectors can be used which promote integration of foreign DNA sequences into the yeast chromosome.

In cases where plant expression vectors are used, the expression of the conjugate coding sequence can be driven by a number of promoters. For example, viral promoters such as the 35S RNA and 19S RNA promoters of CaMV [Brisson et al. (1984) Nature 310:511-514], or the coat protein promoter to TMV [Takamatsu et al. (1987) EMBO J. 6:307-311] can be used. Alternatively, plant promoters such as the small subunit of RUBISCO [Coruzzi et al. (1984) EMBO J. 3:1671-1680 and Brogli et al., (1984) Science 224:838-843] or heat shock promoters, e.g., soybean hsp17.5-E or hsp17.3-B [Gurley et al. (1986) Mol. Cell. Biol. 6:559-565] can be used. These constructs can be introduced into plant cells using Ti plasmid, Ri plasmid, plant viral vectors, direct DNA transformation, microinjection, electroporation and other techniques well known to the skilled artisan. See, for example, Weissbach & Weissbach, 1988, Methods for Plant Molecular Biology, Academic Press, NY, Section VIII, pp 421-463.

Other expression systems such as insects and mammalian host cell systems, which are well known in the art, can also be used by the present invention.

In any case, transformed cells are cultured under effective conditions, which allow for the expression of high amounts of recombinant polypeptide. Effective culture conditions include, but are not limited to, effective media, bioreactor, temperature, pH and oxygen conditions that permit protein production. An effective medium refers to any medium in which a cell is cultured to produce the recombinant conjugate protein of the present invention. Such a medium typically includes an aqueous solution having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

Depending on the vector and host system used for production, resultant proteins of the present invention may either remain within the recombinant cell, secreted into the fermentation medium, secreted into a space between two cellular membranes, such as the periplasmic space in E. coli; or retained on the outer surface of a cell or viral membrane.

Following a predetermined time in culture, recovery of the recombinant protein is effected. The phrase “recovering the recombinant protein” refers to collecting the whole fermentation medium containing the protein and need not imply additional steps of separation or purification. Proteins of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

Proteins of the present invention are preferably retrieved in “substantially pure” form. As used herein, “substantially pure” refers to a purity that allows for the effective use of the protein in the diverse applications, described hereinabove.

It will be appreciated that recombinant production of the conjugates of the present invention can also be effected in-vitro.

In vitro expression can be accomplished, for example, by placing the coding region for the fusion protein in an expression vector designed for in vitro use and adding rabbit reticulocyte lysate and cofactors; labeled amino acids can be incorporated if desired. Such in vitro expression vectors may provide some or all of the expression signals necessary in the system used. These methods are well known in the art and the components of the system are commercially available.

Evaluation of the penetration activity of the conjugates of the present invention is then effected.

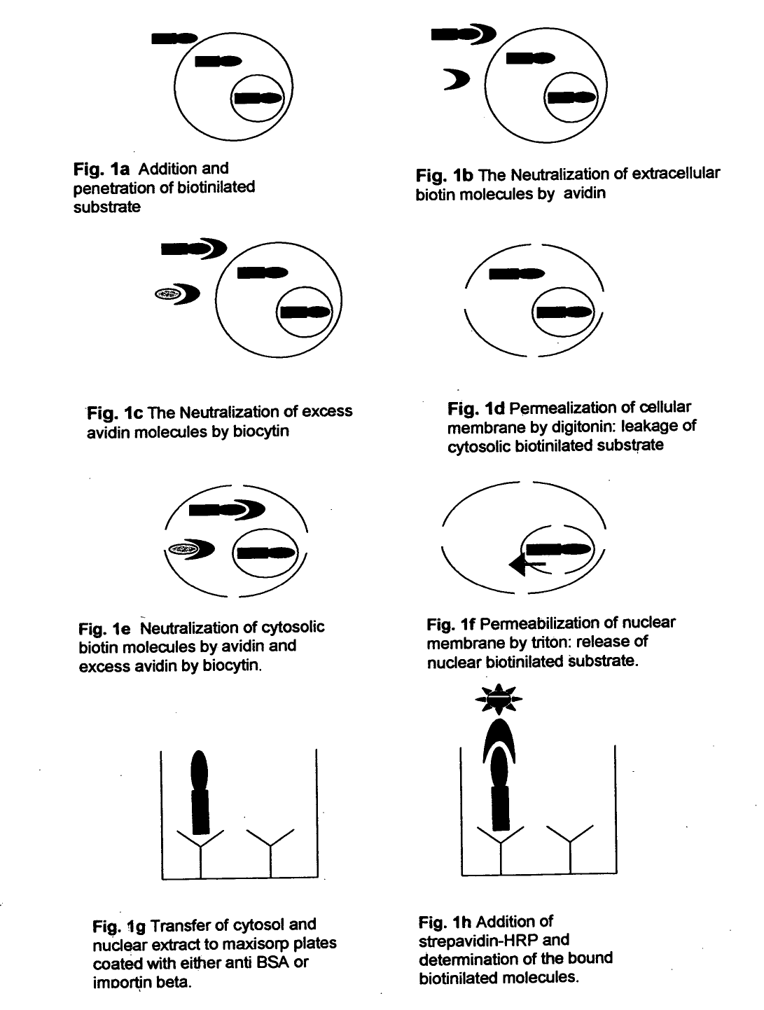

While reducing the present invention to practice, a novel method was developed for quantitatively determining the nuclear uptake and/or the cytoplasmic uptake of a moiety (e.g., a conjugate of the present invention) into cells (see FIG. 1 and the Material and Methods section in the Examples section hereinbelow for further detail). Such a method enables to estimate the amount or concentration of a moiety which penetrates into the cells plasma and/or nuclei, individually and compared one with the other.

Thus, according to another aspect of the present invention, there is provided a method of quantitatively determining the nuclear uptake and/or the cytoplasmic uptake of a moiety.

The method is effected by first contacting the moiety with the cells, so as to enable the penetration of the moiety into the cells. The cells are thereafter fractionated into a cytoplasmic fraction and a nuclei fraction and the amount or concentration of the moiety in each of these fractions is quantitatively determined.

Contacting the moiety with the cells is preferably effected by co-incubating the cells with the moiety.

In order to facilitate the detection of the moiety at the quantitative determining step, the moiety preferably includes a detection group. The detection group is typically attached to the moiety prior to its co-incubation with the cells.

Such a detection group can be, for example, a group that can further form a particular complex with a particular compound that has affinity to this group.

A representative example of a detection group includes biotin. Biotin is a known detection group that typically binds to proteins. The biotin can be detected by, for example, avidin.

Upon contacting the cells and the moiety, and in order to separately determine the cytoplasmic uptake and the nuclear uptake of the moiety, the cell is fractionated into a cytoplasmic fraction and a nuclei fraction. Fractionation is preferably performed by first permeabilizing the cell membrane, without permeabilizing the nuclear membrane, so as to obtain the cytoplasmic fraction and thereafter permeabilizing the nuclear membrane, so as to obtain the nuclear fraction. These permeabilizations are performed using known reagents such as digitonin for permeabilizing the cell membrane and a lysis buffer including Triton for permeabilizing the nuclear membrane.

It should be noted, however, that the method of this aspect of the present invention can be further used to determine the total cellular uptake of a moiety in cells. In such cases, the cells are not fractionated at this stage but are rather permeabilized using a lysis buffer.

Upon fractionating the cells, the moiety's uptake in each fraction is quantitatively determined. Preferred quantitative determination, according to thus aspect of the present invention, is performed as follows:

Each fraction is contacted with a solid phase that has binding affinity to the moiety. The moiety is thereby adhered to the solid phase. A preferred solid phase can be, for example, a microtiter plate, a chip or a glass.

In a preferred embodiment of the present invention, the solid phase includes ELISA plates coated with a substrate that affinity binds the moiety.

Once the moiety is adhered to the solid phases, a detectable molecule that has affinity to the moiety is attached thereto. Such a detectable molecule can be, for example, an enzyme capable of catalyzing a calorimetric reaction, a bead, a pigment, a fluorophore or any other molecule that can be detected and quantified by, for example, calorimetric reaction.

A representative example of a detectable molecule is Horse Reddish Peroxidase (HRP).

The detectable molecule can be directly attached to the moiety or, if it lacks affinity to the moiety, the detectable molecule can be attached to the moiety via another molecule that has affinity to both the moiety and the detectable molecule.

Upon attaching the detectable molecule to the moiety, the detectable molecule is quantitated by, for example, a colorimetric reaction. The amount or concentration of the detectable molecule directly indicates the amount or concentration of the moiety in the measured fraction and hence provides quantitative determination of the uptake of the moiety in the fraction.

Since this method of the present invention can be utilized to quantitatively determine the cytoplasmic and/nuclear uptake of a variety of moieties, it can be utilized as a reliable and efficient tool for comparative measurements. As such, this method of the present invention can be utilized, for example, to compare the uptake of a conjugate with the uptake of its parent compounds; to compare the uptake of the conjugates of the present invention with the uptake of other, known conjugates, etc.

A representative example of the method according to this aspect of the present invention, in which the uptake of a conjugate of the present invention is quantitatively determined, is schematically described in FIG. 1 and includes the following steps:

A conjugate of histone and BSA (a representative example of a protein-of-interest), denoted as squares, labeled by biotin as a detection molecule, denoted as ellipses, is co-incubated with cells.

Avidin (denoted as u-shapes), which forms a complex with biotin, is thereafter added, in order to neutralize the cytoplasmic biotinilated conjugate. In an additional step, biocytin is added so as to neutralize excess of avidin.

The cell membrane is then permeabilized as described above. The cytoplasmic fraction, obtained by this permeabilization, therefore includes the biotinilated conjugate attached to avidin.

Quantitative determination of the cytoplasmic uptake of the conjugate is performed by adhering the above complex to an ELISA plate coated with anti-BSA. Horse Reddish Peroxidase (HRP), as a detectable molecule, is thereafter attached to the avidin molecules in the above complex, and the amount of the HRP is determined by known methods.

Upon permibealizing the cell membrane, the nuclear membrane, which includes the nuclei fraction of the biotinilated conjugate, is permeabilized as described above. The conjugate is adhered to coated ELISA plates, similarly to the cytoplasmic fraction. However, since the HRP lacks affinity to the biotinilated conjugate, an avidin complex of HRP is attached to the adhered biotinilated conjugate. The avidin molecules affinity bind to the biotin and the amount of the HRP is measured, to thereby quantitatively determine the nuclear uptake of the conjugate.

As is mentioned hereinabove, the conjugates of the present invention are preferably utilized in therapeutic approaches which frequently require therapeutic agents to penetrate through both plasma and nuclear membranes.

Hence, according to another aspect of the present invention, there is provided a pharmaceutical composition that comprises, as an active ingredient, the conjugate of the present invention and a pharmaceutically acceptable carrier.

As used herein a “pharmaceutical composition” refers to a preparation of one or more of the conjugates described herein, with other chemical components such as pharmaceutically suitable carriers and excipients. The purpose of a pharmaceutical composition is to facilitate administration of a compound to a subject.

Hereinafter, the term “pharmaceutically acceptable carrier” refers to a carrier or a diluent that does not cause significant irritation to a subject and does not abrogate the biological activity and properties of the administered compound. Examples, without limitations, of carriers are propylene glycol, saline, emulsions and mixtures of organic solvents with water.

Herein the term “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, transdermal, intestinal or parenteral delivery, including intramuscular, subcutaneous and intramedullary injections as well as intrathecal, direct intraventricular, intravenous, intraperitoneal, intranasal, or intraocular injections. Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more pharmaceutically acceptable carriers comprising excipients and auxiliaries, which facilitate processing of the active compounds into preparations which can be used pharmaceutically. Proper formulation is dependent upon the route of administration chosen.

For injection, the conjugates of the invention may be formulated in aqueous solutions, preferably in physiologically compatible buffers such as Hank's solution, Ringer's solution, or physiological saline buffer with or without organic solvents such as propylene glycol, polyethylene glycol. For transmucosal administration, penetrants are used in the formulation. Such penetrants are generally known in the art.

For oral administration, the conjugates can be formulated readily by combining the active compound (i.e., the conjugate or an expression vector encoding a polypeptide conjugate) with pharmaceutically acceptable carriers well known in the art. Such carriers enable the active compound of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by inhalation, the active compound according to the present invention is conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The active compound described herein may be formulated for parenteral administration, e.g., by bolus injection or continues infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active compound in water-soluble form. Additionally, suspensions of the active compounds may be prepared as appropriate oily injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active compound to allow for the preparation of highly concentrated solutions.

Alternatively, the active compound may be in powder form for constitution with a suitable vehicle, e.g., sterile, pyrogen-free water, before use.

The active compound of the present invention may also be formulated in rectal compositions such as suppositories or retention enemas, using, e.g., conventional suppository bases such as cocoa butter or other glycerides.

The pharmaceutical compositions herein described may also comprise suitable solid of gel phase carriers or excipients. Examples of such carriers or excipients include, but are not limited to, calcium carbonate, calcium phosphate, various sugars, starches, cellulose derivatives, gelatin and polymers such as polyethylene glycols.

Pharmaceutical compositions suitable for use in context of the present invention include compositions wherein the active compound is contained in an amount effective to achieve the intended purpose. More specifically, a therapeutically effective amount means an amount of active compound effective to affect symptoms of disease or prolong the survival of the subject being treated.

Determination of a therapeutically effective amount is well within the capability of those skilled in the art, especially in light of the detailed disclosure provided herein.

For any active compound used in the methods of the invention, the therapeutically effective amount or dose can be estimated initially from activity assays in cell cultures and/or animals. Such information can be used to more accurately determine useful doses in humans.

The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Compositions of the present invention may, if desired, be presented in a pack or dispenser device, such as a FDA approved kit, which may contain one or more unit dosage forms containing the active compound. The pack may, for example, comprise metal or plastic foil, such as a blister pack. The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a conjugate of the invention formulated in a compatible pharmaceutical carrier may also be prepared, placed in an appropriate container, and labeled for treatment of an indicated condition. Suitable conditions indicated on the label may include, for example, treatment of a proliferative disorder or disease, a genetic disorder or disease, a bacterial infection and/or a viral infection. Specific disorders, disease or infections treatable by the pharmaceutical composition of the present invention are listed hereinafter.

The conjugates of the present invention can be used in the treatment of a variety of disorders, diseases and infections that require the penetration of a therapeutic ingredient into the cell.

Hence, according to a further aspect of the present invention, there is provided a method of treating a genetic disorder or disease, a proliferative disorder or disease, a bacterial infection and/or a viral infection in a subject in need of such treatment. The method of this aspect of the present invention is effected by administering to the subject, using any route of administration described hereinabove, a therapeutically effective amount, as is defined hereinabove, of a conjugate according to the present invention, which includes a macromolecule-of-interest (e.g., a protein or a nucleic acid) that has therapeutic activity suitable for treating the disorders, diseases and infections delineated hereinabove.

A method of treating a genetic disorder or disease by administration of a therapeutic conjugate that penetrates the cell is referred to in the art as gene therapy. Preferably, such method would be effected by administration of a conjugate that includes a nucleic acid as the macromolecule-of-interest.

Gene therapy may include the addition, the replacement, the deletion, the supplementation, the manipulation and more, of one or more nucleotide sequences in, for example, targeted cells. General teachings on gene therapy may be found in Molecular Biology (Ed. Robert Meyers, Pub VCH, such as pages 556-558). By way of further example, gene therapy can also provide a means by which a nucleotide sequence, such as a gene, can be applied to replace or supplement a defective gene; a pathogenic nucleotide sequence, such as a gene, or expression product thereof can be eliminated; a nucleotide sequence, such as a gene, or expression product thereof, can be added or introduced in order, for example, to create a more favorable phenotype; a nucleotide sequence, such as a gene, or expression product thereof can be added or introduced, for example, for selcetion purposes (e.g., to select transformed cells and the like over non-transformed cells); cells can be manipulated at the molecular level to treat, cure or prevent disease conditions—such as cancer (Schmidt-Wolf and Schmidt-Wolf, 1994, Annals of Hematology 69; 273-279) or other disease conditions, such as immune, cardiovascular, neurological, inflammatory or infectious disorders; antigens can be manipulated and/or introduced to elicit an immune response, such as genetic vaccination.

A method of treating a proliferative disorder or disease by administration of a therapeutic conjugate that penetrates the cell is referred to in the art as cancer therapy. To this end, the conjugates of the present invention may be used to transport into cancer cell molecules that are transcription factors and are able to restore cell cycle control or induce differentiation. For example, it is understood that many cancer cells would undergo apoptosis if a functional P-53 molecule is introduced into their cytoplasm. The conjugates of present invention may be used to deliver such gene products.

For the treatment of a bacterial or viral infection, the method according to this aspect of the present invention can be effected by administering a conjugate in which the macromolecule-of-interest affects antibacterial and antiviral processes. For example, the conjugate of the present invention may be used to transport in the cytoplasm of infected cells recombinant antibodies or DNA binding molecules, which interfere with a crucial step of bacterial and viral replication.

A partial list of disorders and diseases that are treatable by this method of the present invention include: cancer, inflammation or inflammatory disease, dermatological disorders, fever, cardiovascular effects, haemorrhage, coagulation and acute phase response, cachexia, anorexia, acute infection, HIV infection, shock states, graft-versus-host reactions, autoimmune disease, reperfusion injury, meningitis, migraine and aspirin-dependent anti-thrombosis; tumor growth, invasion and spread, angiogenesis, metastases, malignant, ascites and malignant pleural effusion; cerebral ischaemia, ischaemic heart disease, osteoarthritis, rheumatoid arthritis, osteoporosis, asthma, multiple sclerosis, neurodegeneration, Alzheimer's disease, atherosclerosis, stroke, vasculitis, Crohn's disease and ulcerative colitis; periodontitis, gingivitis; psoriasis, atopic dermatitis, chronic ulcers, epidermolysis bullosa; corneal ulceration, retinopathy and surgical wound healing; rhinitis, allergic conjunctivitis, eczema, anaphylaxis; restenosis, congestive heart failure, endometriosis, atherosclerosis or endosclerosis. Cytokine and cell proliferation/differentiation activity; immunosuppressant or immunostimulant activity (e.g. for treating immune deficiency, including infection with human immune deficiency virus; regulation of lymphocyte growth; treating cancer and many autoinimune diseases, and to prevent transplant rejection or induce tumor immunity); regulation of haematopoiesis, e.g. treatment of myeloidor lymphoid diseases: promoting growth of bone, cartilage, tendon, ligament and nerve tissue, e.g. for healing wounds, treatment of burns, ulcers and periodontal disease and neurodegeneration; inhibition or activation of follicle-stimulating hormone (modulation of fertility); chemotactic/chemokinetic activity (e.g. for mobilizing specific cell types to sites of injury or infection); haemostatic and thrombolytic activity (e.g. for treating haemophilia and stroke); anti-inflammatory activity (for treating e.g. septic shock or Crohn's disease); as antimicrobials; modulators of e.g. metabolism or behavior; as analgesics; treating specific deficiency disorders; in treatment of e.g. psoriasis, in human or veterinary medicine. Macrophage inhibitory and/or T cell inhibitory activity and thus, anti-inflammatory activity; anti-immune activity, i.e. inhibitory effects against a cellular and/or humoral immune response, including a response not associated with inflammation; inhibit the ability of macrophages and T cells to adhere to extracellular matrix components and fibronectin, as well as up-regulated fas receptor expression in T cells; inhibit unwanted immune reaction and inflammation including arthritis, including rheumatoid arthritis, inflammation associated with hypersensitivity, allergic reactions, asthma, systemic hapus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with pepticulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or otheroto-rhino-laryngological diseases, dermatitis or other dermal diseases, periodontal diseases or other dental diseases, orchitis or epididimo-orchitis, infertility, orchidaltrauma or other immune-related testicular diseases, placental dysfunction, placental insufficiency, habitual abortion, eclampsia, pre-eclampsia and other immune and/or inflammatory-related gynecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaernic optic neuropathy, excessive scarring, e.g. following glaucoma filtration operation, immune and/or inflammation reaction against ocular implants and other immune and inflammatory-related ophthalmic diseases, inflammation associated with autoinimune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenbam chorea and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumor cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery, bone marrow transplantation or other transplantation complications and/or side effects, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

Additional conditions that are treatable by the method of the present invention are described in WO 98/05635, WO 98/07859 and WO 98/09985, which are incorporated by reference as if fully set forth herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Materials and Experimental Methods

Cultured Cells:

HeLa cell monolayers were grown in DMEM growth medium supplemented with 10% FCS, 0.3 gram/liter L-glutamine, 100 U/ml penicillin and 100 U/ml streptomycin (Beit Haemek, Israel). Cells were incubated at 37° C. in 5% CO₂ atmosphere.

Colo-205 (human colon adenocarcinoma cells; ATCC: CCL 222) were maintained in RPMI 1640 medium, supplemented with 10% FCS, 0.3 gram/liter L-glutamine, 100 U/ml penicillin and 100 U/ml streptomycin (Beit Haemek, Israel). Cells were incubated at 37° C. in 5% CO₂ atmosphere.

Human lymphocytes were obtained from fresh human blood by its fractionation on a ficol gradient, as described in Amos D. B. and Pool P., “HLA typing in Manual of clinical immunology” (N. R. Rose and H. Friedman, Editors) American society for microbiology, Washington D.C. 1976, pp. 797-804.

Buffers:

Transport buffer (TB) comprised 20 mM Hepes pH=7.3, 110 mM potassium acetate, 5 mM sodium acetate, 0.5 mM EGTA, 2 mM DTT, 1 mg/ml leupeptin, 1 mg/ml pepstatin, 1 mg/ml aprotinin and 0.1 mM PMSF.

Synthesis of Peptides:

The Tat-ARM peptide (GRKKRRQRRRPPQC-NH₂; SEQ ID NO:1) and the NLS of the SV40 large T antigen (PKKKRKVC-NH₂; SEQ ID NO:2) were synthesized according to the SPPS method, using an Applied Biosystems Peptide synthesizer model 433A on Rink amide resin (loading 0.65 mmol/gram), by the standard Fmoc chemistry procedure described in Bedford, J. et al. (1992) Int. J. Peptide Prot. Res. 40, 300.

Syntheses of BSA Covalently Coupled with Tat-ARM or the NLS of the SV40 Large T Antigen:

Biotinilated BSA (obtained from Sigma) was coupled with the above peptides according to the procedures described by Melchior et al. [26] and Friedler et al. [25]. In brief, Biotinilated BSA was activated with Sulfo SMCC and was purified on G-25 Sephadex column. The activated protein was mixed with 50-fold excess of pure peptide and incubated at 4° C. overnight. The biotinilated BSA-peptide conjugate was thereafter separated from the free peptide by centrifugation, using vivaspin. The product concentration was determined by the Bradford assay.

Synthesis of Fluorescently-Labeled BSA, CA and Oligonucleotide:

The fluorescently-labeled proteins and oligonucleotide were prepared according to the procedures described in Goldfarb et al. (1986) Nature, 322, 641-644, Friedler et al. [25] and Karni et al. [44]. In a representative example, Lissamine Rhodamine sulfonyl chaloride (4 mg, 7×10⁻³ mmol) and CA (60 mg, 2×10⁻³ mmol) were dissolved in a NaCl/NaHCO₃ buffer, pH=9.6, and the mixture was stirred for 3 hours. The fluorescently-labeled product was thereafter separated on G-25 Sephadex column.

Preparation and Purification of Recombinant Pure Histone Proteins:

Expression plasmids encoding for the individual histones (H2A, H2B, H3 and H4) were kindly obtained from Dr. K. Luger and Dr. T. J. Richmond [24] and were expressed in E. coli strain BL21 (pLysS) and purified as described by Luger et al. [24].

Syntheses of Histone Conjugates:

Conjugates containing histone molecules (either mixture of histones or pure individual histones) covalently linked to the peptides bovine serum albumin (BSA) or carbon adhydrase (CA) or to an oligonucleotide were synthesized according to known procedures [43, 44] of conjugating macromolecules.

The general synthesis method involved the use of the cross-linking reagent Sulfo-SMCC, which covalently bridges between the amino groups of biotinilated- or fluorescently-labeled BSA, CA or oligonucleotide macromolecules and the thiol group of thiolated histones.

Synthesis of a Conjugate Containing a Mixture of Histones Covalently Coupled to Labeled BSA (Histone-BSA Conjugate):

In brief, biotinilated BSA (Sigma) or fluorescently-labeled BSA (prepared as described hereinabove) was activated with Sulfo SMCC and the product was purified on G-25 Sephadex column. The activated BSA was mixed with 20 mg of histones mixture (Sigma, Cat. No. H5505) and the obtained mixture was incubated at 4° C. overnight. The labeled BSA-histones conjugate was thereafter separated from free histone molecules by centrifugation, using vivaspin. The product concentration was determined by the Bradford assay.

Synthesis of a Conjugate Containing H2A or H2B Histone Covalently Coupled to BSA (H2A-BSA conjugate and H2B-BSA Conjugate):

In brief, biotinilated BSA or fluorescently-labeled BSA (prepared as described hereinabove) was activated with Sulfo SMCC as described hereinabove. The activated BSA was mixed with 20 mg of pure H2A or H2B histone (prepared as described hereinabove) and the mixture was incubated at 4° C. overnight. The labeled BSA-histone conjugate was thereafter separated from free histone molecules by centrifugation, using vivaspin. The product concentration was determined by the Bradford assay.

Synthesis of Conjugates Containing Pure Histone or a Mixture of Histones Covalently Coupled to CA:

Fluorescently-labeled CA was prepared as described hereinabove and was activated with Sulfo SMCC. The activated protein was purified on G-25 Sephadex column and was thereafter mixed with 20 mg of histones mixture (Sigma, Cat. No. H5505) or with 20 mg of a pure histone. The mixture was incubated at 4° C. overnight and the fluorescently-labeled CA-histone conjugate was thereafter separated from free histone molecules by centrifugation, using vivaspin. The product concentration was determined by the Bradford assay.

Synthesis of Conjugates Containing Pure Histone or a Mixture of Histones Covalently Coupled to an Oligonucleotide:

Thiol-containing oligonucleotides were obtained from Genetix Pharmaceutic Cambridge, Mass. 02139, USA. The histone-oligonucleotide conjugates were prepared as described hereinabove, by activating the oligonucleotide with Sulfo SMCC and reacting the activated oligonucleotide with a pure histone or a mixture of histones.

Expression and Purification of Importin Beta:

The vector pET28-hIMPb1 was kindly obtained from Dr. V. Citovsky (State University of New-York Stony Brook) and was expressed in E. coli strain BL21(DE3). The histidine (His-Tag; Qiagen) tagged-importin beta fusion protein was expressed and purified by standard protocols following the growth at 37° C. and induction of the E. coli strain at 25° C.

Incubation of a Histones Mixture, of Pure Recombinant Histone Molecules and of Histone Conjugates with Cultured HeLa Cells—Microscopic Observations of Cellular Uptake and Nuclear Import:

A histones mixture containing all five histones (Sigma, Cat. No. H5505) and the four pure recombinant histones molecules (H2A, H2B, H3 and H4) were labeled with Lissamine Rhodamine (Molecular Probes) or covalently attached to fluorescently (Lissamine Rhodamine) labeled BSA, CA or oligonucleotide molecules, using a well known method for labeling molecules with Rhodamine [25].

For incubation with histone molecules, HeLa cells (3×10⁴ cells per coverslip) were cultured on 10 mm coverslips to subconfluent density. After the removal of the culture medium, the cells were washed three times with TB and then exposed to various concentrations of labeled histone preparations or labeled histones conjugates, labeled as described hereinabove, at 37° C. or at 4° C. At the end of the incubation period the cells were washed three times with TB and in some experiments were observed directly thereafter by fluorescent microscopy. In most of the experiments, the cells were fixed in 4% (v/v) formaldehyde dissolved in TB. The fixed cells were examined by fluorescence microscopy (Zeiss Germany, a 40× objective; Apoplan) or by confocal microscopy using an MRC 1024 confocal imaging system (Bio-Rad). The microscope (Axiovert 135M; Zeiss Germany, a 63× objective; Apoplan; NA 1.4) was equipped with an argon ion laser for Rhodamine excitation at 514 nm (emission 580).

Nuclear Import of Histone Molecules and Histone Conjugates in Permeabilized HeLa Cells:

The nuclear import of fluorescently-labeled histone molecules and histone conjugates was performed according to the procedure described in Friedler et al. [43] and Karni et al. [44] for nuclear import of fluorescently-labeled BSA-NLS conjugates.

Nuclear Import of Histone Molecules and Histone Conjugates Following Microinjection:

Histone molecules and histone conjugates were microinjected into intact cells exactly as described in Graessmann M. and Graessmann A. (1983), Methods Enzymol. 101, 482-92.

A Novel Assay for Quantitative Estimation of Histone Molecules and/or Histone Conjugates Within the Cytosol and Nuclei of Intact Colon Cells:

A novel assay for quantitatively estimating the cytoplasmic accumulation and nuclear import of externally added histones has been developed and is schematically presented in FIG. 1.

Intact colon cells (15-20×10⁵ cells) in TB were incubated with either biotinilated histones, the four pure recombinant histones molecules (H2A, H2B, H3 and H4; 0.1 mg/ml in TB) or with biotinilated histone conjugates (1 mg/ml in TB), in a final volume of 60 ml for 1 hour, at 37° C. or at 4° C. Histone molecules were conjugated to biotin maleimide or covalently attached to biotinilated-BSA (sigma) as described above [25]. At the end of the incubation period, 200 ml of TB were added and the extracellular transport substrate was removed by centrifugation of the cell suspension for 5 minutes at 100 rpm. After removal of the supernatant, the cells in the pellet were suspended in 100 ml of avidin in TB (1 mg/ml) (see, FIG. 1), in order to neutralize the remaining extracellular biotinilated histones or conjugates. After 30 minutes incubation at 37° C., unbound avidin was deactivated by the addition of 100 ml of Biocytin in TB (2 mg/ml). Following another 15 minutes of incubation the samples were centrifuged as above and the supernatant was removed.

Permeabilization, usually as was observed by phase microscopy, was performed using 30 ml of digitonin solution (0.08 mg/ml), completed within 30 seconds at 37° C. and was terminated by the addition of 200 ml TB. The samples were centrifuged and the supernatant, containing the cell cytosols, was removed and stored in the cold. The remaining extranuclear biotinilated transport substrates were neutralized by the addition 100 ml of avidin in TB (0.1 mg/ml). After 30 minutes incubation at 4° C., 100 ml of Biocytin in TB (0.2 mg/ml) were added and the samples were incubated for another 15 minutes at 4° C. The samples were centrifuged as above, 200 ml of the supernatant were removed and the nuclei in the pellet were lysed by the addition of 200 ml of lysis buffer (1% Triton X-100 in PBS) (see, FIG. 1). Following vigorous mixing, lysis was completed by incubation overnight at 4° C. For estimation of cellular accumulation (cytosol and nuclei), the cells were lysed by 200 ml of lysis buffer and not by digitonin. Evaluation of biotinilated molecules within the cellular lysate was performed as described for estimation of biotinilated molecules within the nuclei lysate (see, [26] and FIG. 1). Briefly, the biotinilated BSA-histone conjugates present in the cytosol and in the nuclei lysate were quantitatively estimated following binding to anti-BSA coated plates and by the use of Avidin-HRP, as described by Melchior et al. [26]. Biotinilated histones were attached to importin beta-coated plates essentially as described by Feinberg et al. [in preparation]. For coating, a solution of importin beta (4.3 mg/ml in NaHCO₃/Na₂CO₃ buffer pH=9.6) was added to 96 maxisorp plates (Nunc Inc.) and were incubated over night at 4° C. All the subsequent steps of the addition of Avidin-HRP and its estimation were performed as described above. The presented results are an average of triplicate determinations.

Effect of the Histones on Cell Viability:

To study the effect of the peptides on cell viability, increasing concentrations of the peptides were added to cultured cells (96 well 3×10⁴ cells per well in DMEM). Following incubation at 37° C. for 30 minutes, 100 ml of Tryphan Blue solution was added (0.4% in HBSS buffer; Sigma), and HBSS buffer (5:3) and viable cells were counted after 5 minutes of continuous stirring. The results indicate that the cell death was less than ±20%.

### Experimental Results

Penetration of Histones Mixture into Cultured Cells Cytoplasm and Nuclei—Microscopic Observations:

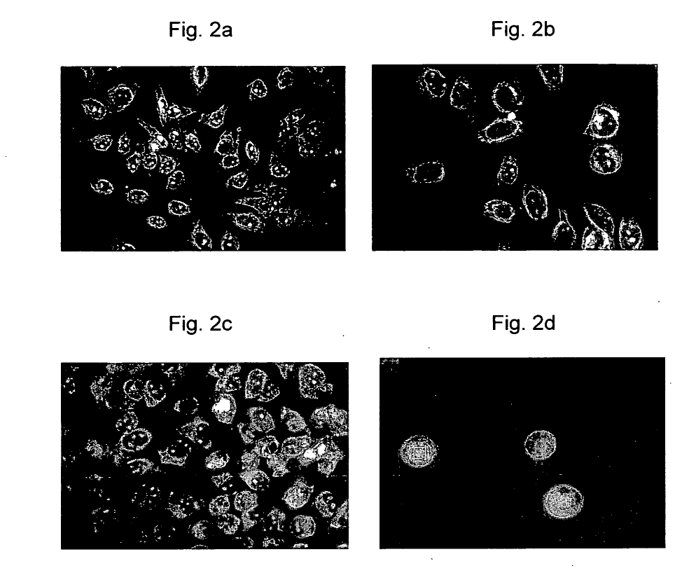

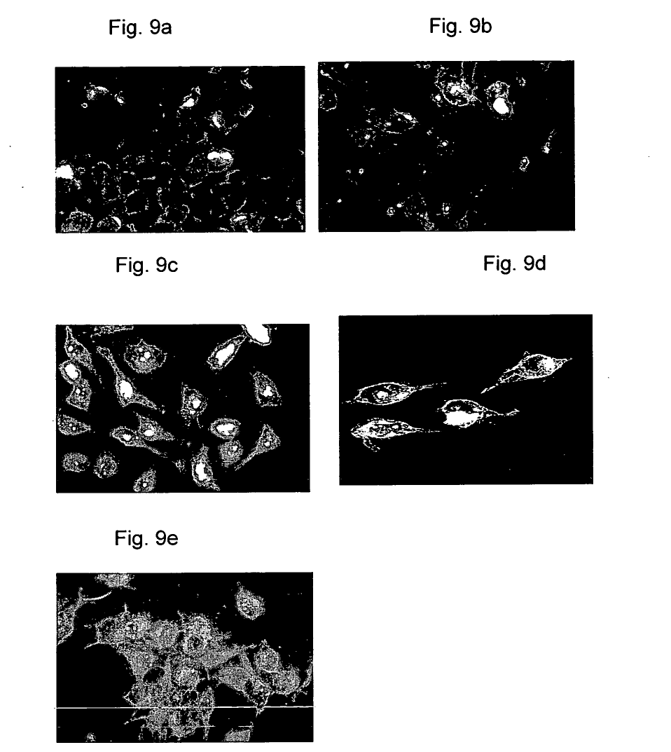

The microscopic observations are presented in FIGS. 2a-d and in Table 3 hereinbelow. The microscopic images presented in FIGS. 2a-c demonstrate that histone proteins are able to penetrate into HeLa cultured cells and to accumulate within their nuclei. As is shown in FIG. 2a, incubation of a fluorescently-labeled mixture containing the five individual histones (H1, H2A, H2B, H3 and H4) with HeLa cultured cells for 1 hour at 37° C. resulted in extensive fluorscent staining of both the cells cytoplasm and the intranuclear space. It should be noted that appearance of fluorescent molecules within the cells cytoplasm and nuclei could be detected already after 15 minutes of incubation at 37° C. As is shown in FIG. 2b, incubation of the histones mixture with HeLa cells at 4° C. also resulted in appearance of fluorescent molecules within the cells cytoplasm with very little, if any, fluorescence within the cells nuclei. As is shown in FIG. 2c, excess (50:1, mole/mole) of non-labeled histones inhibit only the nuclear import of the histones but did not affect their translocation into the cells cytoplasm. As is shown in FIG. 2d, the labeled histones were also able to penetrate human lymphocytes. It should be noted that the same results, namely accumulation of fluorescently-labeled histones within HeLa cells cytosol and nuclei, have been observed also in unfixed cells, thus excluding the possibility that the observed cellular uptake and nuclear import resulted from the fixation procedure [27]. Although the appearance of the labelled histones within the nuclei and especially in the nucleoli indicates, by itself, that the histones are localized within the cells and are not surface bound, it should be further noted that the same results have been observed by the confocal microscope (see, for example, FIGS. 9b and 9d).

The intracellular accumulation of externally added histones within cultured cells has been described before [16, 18]. However, previous studies have indicated that the intracellular accumulation of externally added histones is attributed mainly to endocytosis, resulting in enclosure of the histone molecules within endocytic vesicles. In sharp distinction, the results presented herein (see, FIG. 2 and Table 3) clearly indicate that the accumulation of the externally added histones within the HeLa cells is not due to endocytosis but is attributed to a direct translocation of the histone proteins across the cells plasma membranes, as is concluded from the results showing fluorescent staining in cells incubated with histones at 4° C. (FIG. 2b) or in the presence of a high excess of unlabelled histones (FIG. 2c).

TABLE 3

| Experimental | Histone-mixture | | LDL | LY | Tat-ARM |
|---|---|---|---|---|---|
| conditions | Nuclei | Cytosol | Cytosol | Cytosol | Cytosol |
| Control | + | + | + | + | + |
| DNP (1 mM) + | − | + | N.D. | N.D. | + |
| NaF (2 mM) + | | | | | |
| Iodoacetic | | | | | |
| acid (1 mM) | | | | | |
| Colchicine | + | + | − | + | + |
| (20 μM) | | | | | |
| Brefeldin A | + | + | N.D. | N.D. | + |
| (10 μM) | | | | | |
| Nystatin | + | + | − | − | N.D. |
| (50 μg/ml) | | | | | |
| Cytochalasin | + | + | − | + | N.D. |
| D (5 μM) | | | | | |
| Chloroquine | + | + | − | − | N.D. |
| (50 μM) | | | | | |
| Nocodazole | + | + | − | + | N.D. |
| (20 μM) | | | | | |
| Sucrose | + | + | N.D. | N.D. | + |
| (0.5 M) | | | | | |

N.D. = not determined

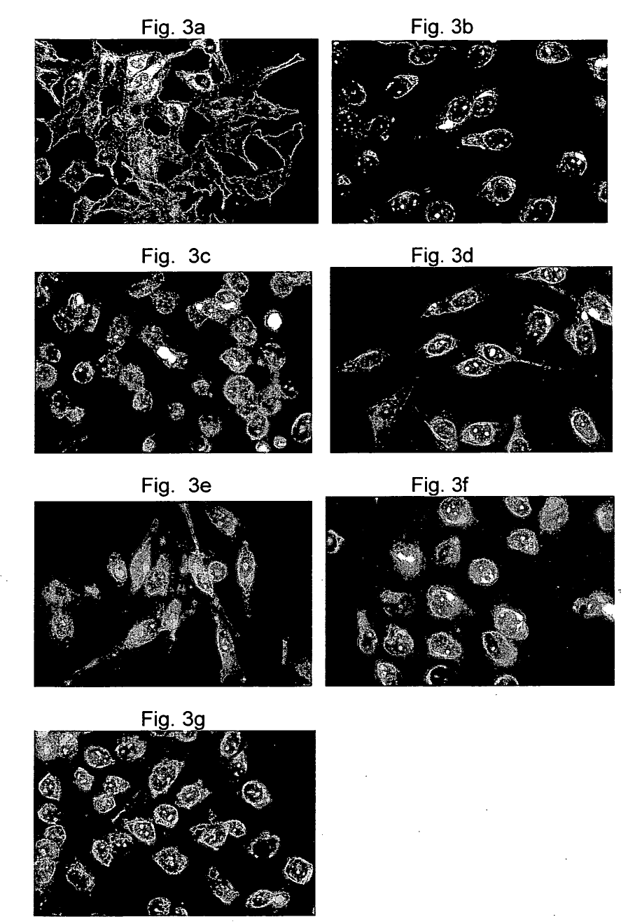

FIG. 3 presents the microscopic observations of intracellular accumulation of histones in ATP depleted cells and in the presence of various inhibitors that affect endocytosis. These microscopic observations, which are also summarized in Table 3, unequivocally demonstrate that the histone molecules are not taken into the HeLa cells by endocytosis but directly penetrate cells plasma membranes. FIG. 3a presents the micrograph observed following incubation of labeled histones with ATP depleted cells and clearly indicates that the labelled histones were able to penetrate into the cytosol of the ATP depleted cells. Though, only the cytoplasm of these cells appeared fluorescent while the nuclei remained dark with no fluorescent staining. Accumulation within the cytosol appears to be an ATP independent process but the translocation into the nuclei is, as expected, energy dependent [28]. FIGS. 3b-f present the micrographs obtained following incubation of labeled histones and HeLa cells in the presence of a battery of inhibitors which effect, directly or indirectly, internalisation via endocytosis or intracellular trafficking, namely colchicine [29] (FIG. 3b), cytocalaszin D [30] (FIG. 3c), BFA [30] (FIG. 3d), nystatin [31] (FIG. 3e) and nocadozole [32] (FIG. 3f). The microscopic observations show that the accumulation of the fluorescent molecules within the cells cytoplasm and nuclei are the same both in the presence or absence of these inhibitors. As is shown in FIG. 3g, the cells cytoplasm and nuclei were highly fluorescent also in cells incubated in a medium containing 0.5 M of sucrose. Such conditions were shown to cause complete blockage of the endocytosis process [31].

In order to ensure that the various inhibitors used in the experiments above block endocytosis and/or pinocytosis under the experimental conditions used, their effects on the uptake of fluorescently labeled LDL (Di1-AC-LDL) and of LY (Lucifer Yellow), which are known to be taken into cells by endocytosis and pinocytosis respectively [29, 30], were studied. The effect of these inhibitors on the ability of a synthetic peptide bearing the Tat-ARM sequence to penetrate into the cultured HeLa cells was also studied. The Tat-ARM peptide is known as a peptide that directly penetrates cells plasma membranes [22]. The results are summarized in Table 3 hereinabove and clearly show that all the four inhibitors used, namely colchicine [34], Cytochalasin D, Nocadozole and nystatin, completely blocked uptake of the LDL molecules and hence prove that the inhibitors used are functional and active under the tested conditions. In addition, nystatin completely inhibited pinocytosis of the LY molecules [33]. However, no inhibition was observed on the penetration of the Tat-ARM peptide, as was expected upon published reports [22]. These results clearly indicate that the histone molecules, when added to cells treated with the four inhibitors, behave similar to the Tat-ARM peptide and are clearly distinct from the LDL complex or the LY molecules. Table 3 and FIG. 3d further show that treatment of HeLa cells with Chloroquine or BFA did not have any effect on the ability of histone molecules to penetrate into these cells. In view of the above results, it is un-avoided to conclude that histone molecules can translocate the HeLa cells plasma membrane.

Quantitative Determination of the Penetration of Histone Molecules into Cells Cytoplasm and Nuclei (Measured by the Novel Quantitative Assay System of the Present Invention):

The penetration of histone molecules into the cytoplasm and nuclei was further measured by the novel quantitative assay of the present invention, which is described in detail hereinabove, in the Materials and Methods section, and is further illustrated in FIG. 1.

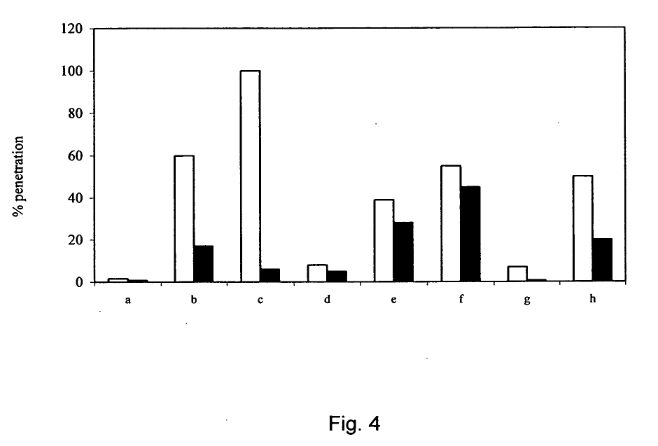

The obtained results are presented in FIG. 4 (a bar graph) and indicate that following incubation of externally added biotinilated histones with colon cells, the histone molecules penetrated the cells while most of the intracellular histones accumulated within the cells nuclei and only about 10% of the histone molecules remained within the cells cytosol (see, FIG. 4, bar c).

The quantitative assay of the present invention further provides confirmation for previous results, based mainly on microscopic observations [22], with respect to the cell penetration of biotinilated Tat-ARM. As is shown in FIG. 4, bar b, most of the biotinilated Tat-ARM accumulated within the cells nuclei, as in the case of the histone molecules. However, the quantitative assay teaches that the penetration ability of the histone molecules is better than that of the Tat-ARM peptide (see, FIG. 4, bars b and c).

As is shown in FIG. 4, bar a, unlike the histone molecules and the Tat-ARM, externally added biotinilated BSA molecules failed to penetrate into the colon cells, as the recipient cells as well as their plasma membrane were intact under the experimental conditions used.

The results depicted in FIG. 4, bar d, confirm the specific binding of histone molecules to importin beta coated plates, as it is shown that very little, if any, histone molecules were attached to uncoated plates. The requirement for a functional plasma membrane is further inferred from the obtained results, as FIG. 4, bar g, clearly indicate that very little penetration of histone molecules occurred with formaldehyde fixed cells.

The quantitative assay of the present invention further confirms the microscopic studies summarized in FIG. 2 as it demonstrates that histones molecules are able to penetrate into intact cells also at 4° C. (FIG. 4, bar e). However, the quantitative assay further indicates that under these conditions the translocation into the cells nuclei was inhibited and relatively higher amounts of histones were found in the cells cytoplasm, as compared to the cells penetration at 37° C. (FIG. 4, bar c). Comparing the total amount of histone molecules that penetrated both the cytoplasm and the nuclei at 37° C. to that penetrated at 4° C., reveals a reduction of only 30% under the later conditions.

Similar results were obtained following incubation of the histone molecules with ATP depleted cells (FIG. 4, bar f), again strengthening the view that the penetration is energy independent. In ATP depleted cells, the relative amount of the intracellular histone molecules was reduced in the nuclei and increased in the cytosol, similar to cells incubated at 4° C., indicating, as expected, inhibition of nuclear import [35] under these conditions.

As is shown in FIG. 4, bar h, almost identical results were obtained when the penetration of labelled histones was studied in the presence of ×100 (mole/mole) excess of unlabelled histone molecules, demonstrating that the unlabelled histones did not compete with the penetration of the labelled ones. A decrease of only 30% was observed in the total amount of the intracellular histones in the cells, with a relative change in the nuclei/cytoplasm ratio, indicating inhibition of nuclear import of the labelled histones under these conditions. The obtained quantitative results indicate that the externally added unlabelled histones penetrate into the colon cells similar to the labelled histones and thus inhibit the translocation of labelled histone into the cells nuclei. A decrease of 50% in the intranuclear histones level has been observed under these conditions while the relative amount of the histones in the cytosol was increased. However, the results indicate that the externally added unlabelled histones caused very little inhibition, if any, on the overall penetration process.

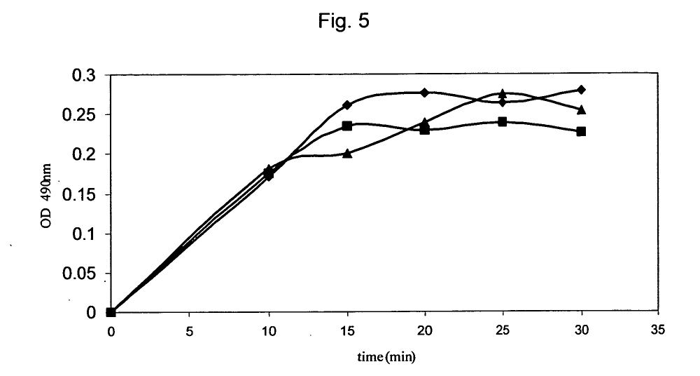

FIG. 5 presents the results obtained by kinetics studies, which further strengthen the view that the histones are not taken into the colon cells via an endocytic process. As is shown in FIG. 5, the same amount of histone molecules penetrated into the cells at 37° C. and at 4° C., reaching a maximum value following 15 minutes of incubation at both temperatures. Furthermore, almost the same kinetics was observed following addition of histone molecules to colon cells incubated in the presence of 0.5 M sucrose. A decrease of only 25% in the total amount of the intracellular histones was observed under these conditions, which are known to totally inhibit uptake by the endocytic pathway [31].

Penetration of Individual Histones into Cells Cytoplasm and Nuclei:

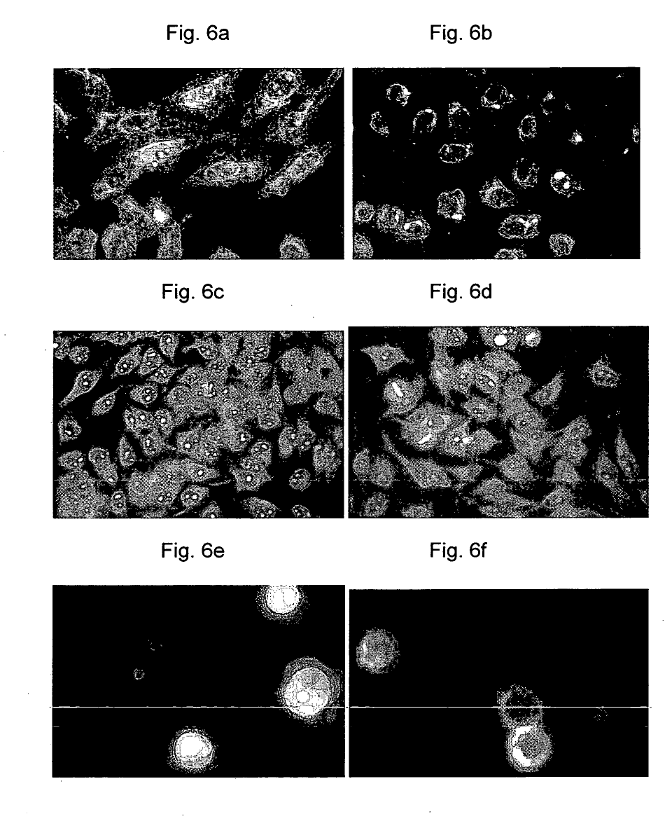

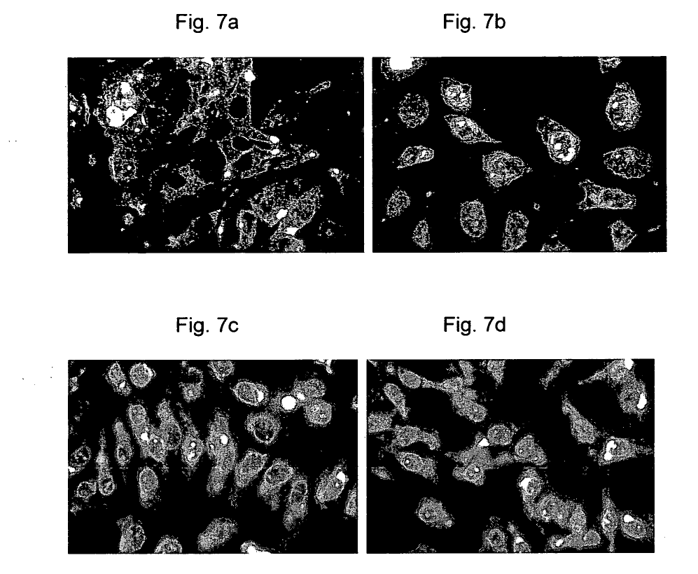

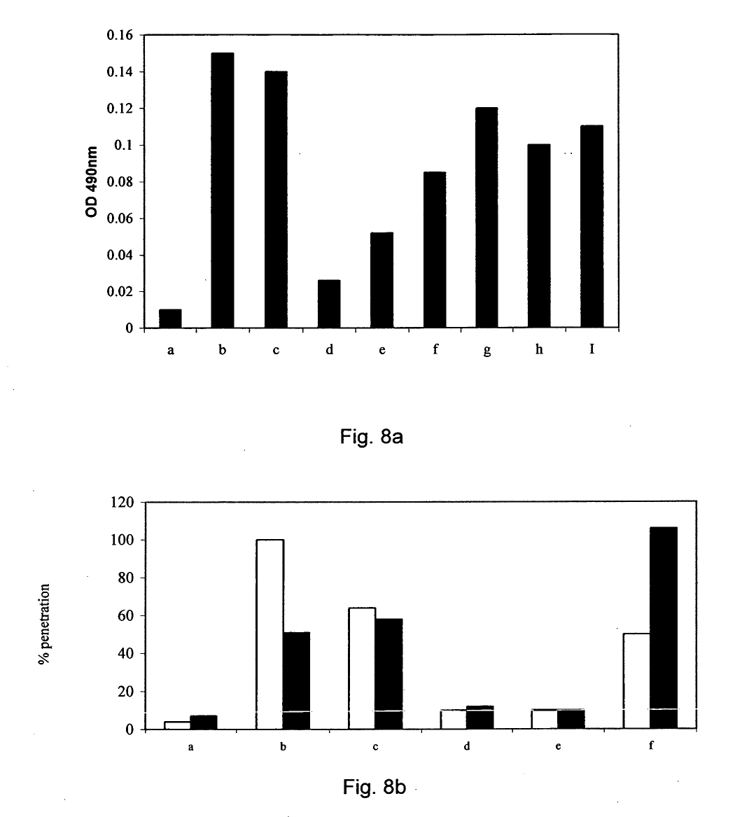

As is well established in the art, individual histone proteins tend to form complexes among themselves [10]. Hence, as the experimental results described hereinabove relate to the penetration of a mixture of all the five histones, there was an interest to study whether each of the five individual histones is able to penetrate intact cells and to what extent, to thereby exclude or conclude that the penetration is attributed only to certain histone complexes. FIGS. 6-8 present the results obtained with respect to the penetration of the various individual histones. The micrographs depicted in FIGS. 6a-f show that the histone H2A readily penetrates into the cell cytosol and nuclei whereas the histone H2B penetration into the cell is low and occurs mainly into the cells cytosol. These observations were confirmed by the quantitative assay results, depicted in FIGS. 8a-b. As is shown in FIG. 8a (a bar graph), the amount of the intracellular histone H2A (bar b) was very close to that found following incubation of the cells with the histones mixture (bar c). On the other hand, the penetration of histone H2B was lower (bar d). Addition of unlabelled H2A to labelled H2B increased the penetration of the later (bar e). This trend is further demonstrated in FIG. 6d. As is shown in FIG. 7 and is further confirmed quantitatively (FIG. 8a, bars f-i), the penetration extent of the histones H3 and H4 was similar but somewhat lower than that of the H2A or the histones mixture. Most of the intracellular H3 was accumulated within the cytoplasm with very little if any in the intranuclear space (FIG. 7a). When a combination of the two histones H3 and H4 was used, their penetration extent was always higher than that of the individual histones (FIGS. 7a-d).

In experiments conducted with individual histones in the presence of the various inhibitors described hereinabove, with respect to the histones mixture (see, Table 3), it was found that the inhibitors had no effect on the penetration ability of the individual recombinant histones. The cell penetration of various individual histones incubated at 4° C. with HeLa cells was also observed and showed no particular change in the penetration extent thereof.

Since H2A exhibited the highest penetration activity, it was used in additional quantitative determinations, presented in FIG. 8b. These additional quantitative results demonstrate that the intracellular distribution of H2A is very similar to that observed with the histones mixture (see, FIG. 4). In control intact cells the large majority of the intracellular H2A molecules was accumulated within the nuclei (FIG. 8b, bar b), while in ATP depleted cells the H2A molecules were equally distributed between the nuclei and the cytosol (FIG. 8b, bar c). However, the total intracellular amount of the H2A in ATP depleted cells was close to that found in control untreated cells, again indicating that the penetration process is energy independent. The same results were observed when ×50 excess of unlabelled H2A was added (FIG. 8b, bar f), namely very little, if any, change was observed in the total amount of the intracellular H2A but a significant alteration occurred in its nuclear:cytosol ratio.

Cellular Uptake and Nuclear Import of Covalently Attached Histone-BSA Molecules:

The cellular uptake and nuclear import of histone molecules that covalently couple BSA was measured in order to determine the ability of the histone molecules to deliver macromolecules such as proteins into living cells. The results are depicted in FIGS. 9-11 and demonstrate that the mixture of the histones as well as the pure histone H2A were able to mediate the penetration of covalently attached BSA molecules into intact cells.

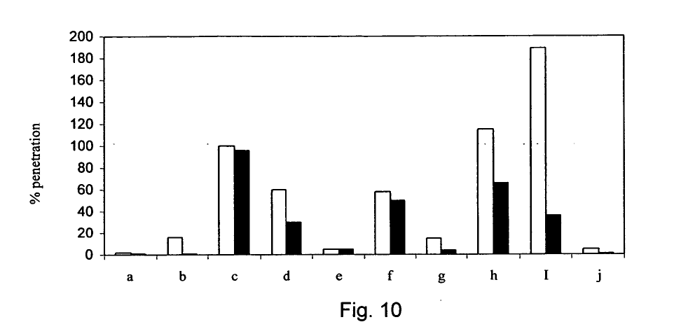

As is shown in FIGS. 9a and 10, the fluorescently labelled (FIG. 9a) as well as the biotinilated (FIG. 10, bar a) unattached BSA molecules were impermeable. These results clearly prove the intactness of the cell plasma membrane toward BSA. It should be noted that in the experiments conducted with the histone-BSA conjugates, the BSA molecules were labelled and therefore the appearance of intracellular fluorescence or biotin labelled molecules clearly indicated the presence of BSA molecules.

As is shown in FIG. 9b, the intracellular histone-BSA conjugates (conjugates containing mixture of histones covalently coupled to BSA) were equally distributed between the cytosol and the nuclei. Penetration into the cells, and not absorption by the cells surface, as well as translocation into the nuclei, of the histone-BSA conjugate, was confirmed by using confocal microscopy (FIG. 9d) and the quantitative assay system (FIG. 10).

Microscopic observations (FIG. 9c) and the quantitative assay (FIG. 10, bar i) indicated, surprisingly, that the addition of non-labelled histones mixture (×50) to the labelled histone-BSA conjugates greatly stimulated the penetration of the conjugate.

The specific histone-mediated penetration of the BSA molecules was further inferred from the results depicted in FIG. 10 (bar b), which show that BSA molecules bearing the NLS of the large SV40 T antigen [36] were practically impermeable.

The quantitative results depicted in FIG. 10 further show a reduction of about 40-50% in the penetration extent of the histone-BSA conjugates into cells incubated at 4° C. (bar d) or into ATP depleted cells (bar f). As the histones cellular uptake in ATP-dependent, it is conceivable that a small amount of the added histones is taken into the cells by endocytosis and that this fraction is larger in the case of histone-BSA conjugates.

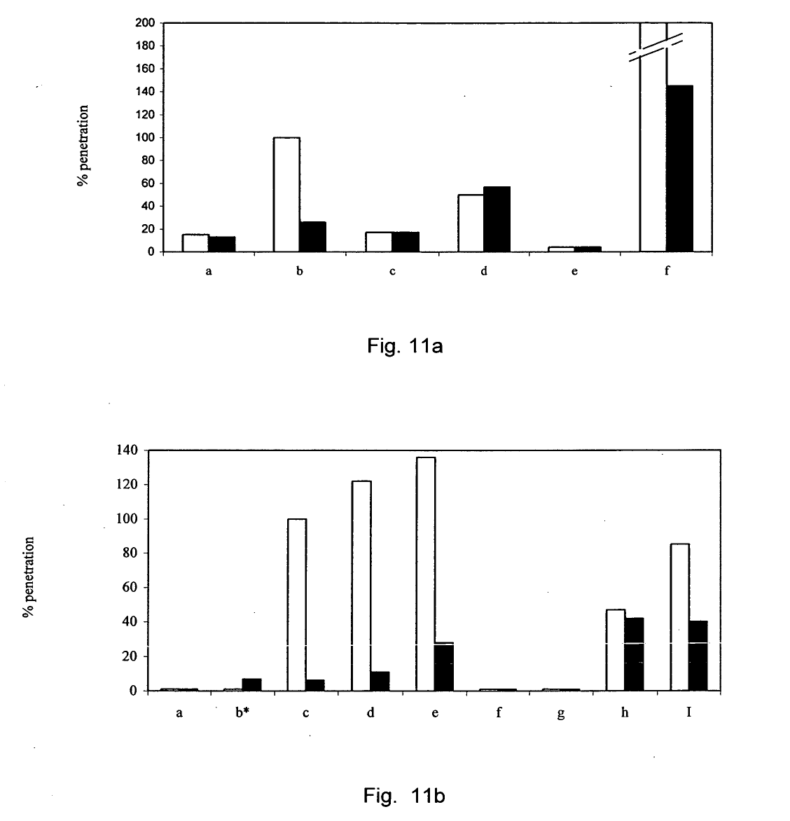

As is depicted in FIG. 11a, essentially the same results were obtained when histone H2A-BSA conjugates were used. However, it was found that, similar to what has been observed with H2A itself, most of the H2A-BSA conjugate accumulated within the intranuclear space. It was further found that addition of excess unlabelled H2B greatly stimulated the penetration of the labelled conjugate (FIG. 1a, bar f).

The experimental results depicted in FIG. 11b showed that as in the case of H2B (see, FIG. 6a), the biotinilated H2B-BSA conjugate hardly penetrated into the recipient cells. However, as is shown in FIG. 11b, bars b and e, the addition of non-biotinilated H2A greatly stimulated the penetration of the biotinilated H2B-BSA conjugate and, interestingly, most of the intracellular biotinilated H2B-BSA conjugates have been translocated into the cells nuclei. Very little inhibition in the penetration process has been observed following incubation of a mixture containing biotinilated H2B-BSA conjugate and non-biotinilated H2A with cells incubated at 4° C. (FIG. 11b, bar i) or with ATP depleted cells (FIG. 11b, bar h).

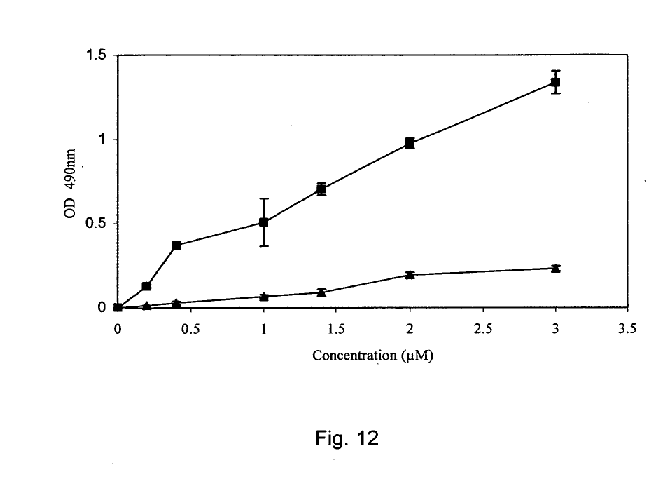

FIG. 12 depicts the results obtained when increasing concentrations of histone-BSA conjugate were used. These results show that under the experimental conditions used, saturation has not been reached, again indicating that the majority of the conjugated molecules are directly translocated through the cell plasma membrane. The quantitative results depicted in FIG. 12 further show that while a synthetic peptide bearing the Tat-ARM NLS sequence [22] was also able to mediate the penetration of covalently attached BSA molecules, the ability of the histone molecules to mediate cell penetration of BSA was about 5 times higher, at all the concentrations measured, as compared with that of the Tat-ARM.

Effect of the Histones on Cell Viability:

The results obtained in the studies on the effect of the histone molecules on cell viability indicated that the cell death was less than ±20%.

Nuclear Import of Histone-BSA Conjugates and Histone-CA Conjugates into Permeabilized Cells, Microinjected Cells and Cultured Cells:

The cellular uptake and nuclear accumulation of the flourescently-labeled histone-BSA and histone-CA conjugates, prepared as described hereinabove, was followed by fluorescence microscopy.

Control experiments clearly showed that neither the BSA nor the CA molecules are able to penetrate into intact cells and accumulate within their nuclei.

The nuclear import of the labeled histone conjugates was first followed in permeabilized HeLa cells, under known experimental conditions [43, 44] and was characterized by all the features that characterize active nuclear import, namely ATP dependent and inhibition by Wheat Germ Agglutinin (WGA), by GTP-γ and by unlabeled histones. The results are summarized in Table 4 below and clearly indicate active nuclear import of the conjugates. (+ indicates that most of the nuclei in the microscopic fields are highly fluorescent; − indicates no fluorescence in the nuclei; +/− indicates that most of the nuclei are very weakly fluorescent).

TABLE 4

| Experimental Conditions | FL-BSA-Histones | FL-CA-Histones |
|---|---|---|
| + Reticulocite Extract | ++ | − |
| − Reticulocite Extract | ++ | ++ |
| 4° C. | − | − |
| WGA | +/− | − |
| GTP-γ- | − | − |
| ATP depletion | − | − |

In experiments conducted in cultured HeLa cells, both the histone-BSA conjugates and the histone-CA conjugates showed fast accumulation within the cytosol and nuclei of the cultured cells. The penetration extent of the histone-CA conjugates was higher than that of the BSA conjugates

Nuclear accumulation of the histone-BSA and the histone-CA conjugates, in the same ratios, has been further observed following microinjection experiments.

Cellular Uptake and Nuclear Import of Histone-Oligonucleotide Conjugate:

An oligonucleotide antisense that induces preferential degradation of the acetylcholine esterase enzyme (AchE) [42] has been fluorescently labeled and covalently linked to histone molecules, as described hereinabove. Microscopic observations clearly showed that incubation of the labeled oligonucleotide-histone conjugate with cultured HeLa cells resulted in the penetration and accumulation of the conjugate within the cells cytoplasm, indicating that histone molecules can serve as an efficient carrier also for oligonucleotides and are therefore able to translocate oligonucleotides into living cells.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES CITED BY NUMERALS

### Additional References are Cited in the Text

1. Uherek, C., and Wels, W. (2000) Advanced Drug Delivery Rev 44, 153-166
2. Fender, P., Ruigrok, R. W., Gout, E., Buffet, S., and Chroboczek, J. (1997) Nat Biotechnol 15, 52-56
3. Wagner, E. (1998) in Self assembling complexes for gene delivery (Kabanov, A. V., Felgner, P. L., and Seymour, L. W., eds), pp. 309-322, Wiley, Chichester
4. Boussif, O., Lezoualch, F., Zanta, M. A., Mergny, M. D., Scherman, D., Demeneix, B., and Behr, J. P. (1995) Proc Natl Acad sci USA 92, 7297-7301
5. Felgner, P. L., Barenholz, Y., Behr, J. P., Cheng, S. H., Cullis, P., Huang, L., Jessee, J. A., Seymour, L., Szoka, F. C., Thierry, A. R., Wagner, E., and Wu, G. (1997) Hum Gene Ther 8, 511-512
6. Zenke, M., Steinlein, P., Wagner, E., Cotton, M., Beug, H., and Birnstiel, M. L. (1990) Proc Natl Acad sci USA 87, 4033-4037
7. Johnson-Saliba, M., Siddon, N., Clarkson, M., Tremethick, D., and Jans, D. (2000) FEBS letters 467, 167-174
8. Van-Holde, K. (ed) (1989) Chromatin, Springer, N.Y.
9. Baake, M., Doenecke, D., and Albig, W. (2001) J Cell Biol 81, 333-346
10. Luger, K., Mader, A. W., Richmond, R. K., Sarget, D. F., and Richmond, T. J. (1997) Nature 389, 251-260
11. Fritz, J., Herweijer, H., Zhang, G., and Wolff, J. (1996) Hum Gene Ther 7(12), 1395-1404
12. Bottger, M., Zaitsev, S. V., Otto, A. Haberland, A., and Vorob'ev, V. (1998) Bioch et Biophys Acta 1395, 78-87
13. Balicki, D., Reisfeld, R. A., Pertl, U., Beutler, E., and Lode, H. N. (2000) Proc Natl Acad sci USA 97(21), 11500-11504
14. Chen, J., Stichles, R., and Daichendt, K. (1994) Hum Gene Ther 5, 429-435
15. Balicki, D., Putnam, C. D., Scaria, P. V., and Beutler, E. (2002) Proc Natl Acad sci USA 99(11), 7467-7471
16. Ryser, H. J. P., and Hancock, R. (1965) Science 150, 501-503
17. Brix, K., Summa, W., Lottspeich, F., and Herzog, V. (1998) J Clin Invest 102(2), 283-293
18. Murphy, R. F., Jorgensen, E. D., and Cantor, C. R. (1982) J Biol Chem 257(14), 1695-1701
19. Gariepy, J., and Kawamura, K. (2001) Trends Biotechnol 19(1), 21-28
20. Kuismanen, E., and Saraste, J. (1989) Methods Cell Biol 32, 257-274
21. Vives, E., Charneau, P., VanRietschoten, J., Rochart, H., and Bahraoui, E. (1994) J. Virol. 68, 3343-3353
22. Vives, E., Brodin, P., and Lableu, B. (1997) Journal of Biological Chemistry 272 (25 June 20), 16010-16017
23. Higashhijima, T., Burnier, J., and Ross, E. M. (1990) J. Biol. Chem. 265, 14176-14186
24. Luger, K., Rchsteiner, T., and Richmond, T. (1999) Methods Mol Biol 119, 1-16
25. Friedler, A., Luedtke, N., Friedler, D., Loyter, A., Tor, Y., and Gilon, C. (2000) J. Biol. Chem. 275, 23783-9
26. Melchior, F., Paschal, B., Evance, J., and Gerace, L. (1993) J. Cell. Biol. 123(6), 1649-1659
27. Lundberg, M., and Johansson, M. (2002) bioch et Biophys Res Com 291, 367-371
28. Scmid, S., and Carter, L. (1990) J Cell Biol 111, 2307-2318
29. Skrzypek, E., Cowan, C., and C.Straley, S. (1998) Molecular Microbiology 30(5), 1051-1065
30. Elliott, G., and O'Hare, P. (1997) Cell 88(2), 223-33
31. Okamoto, Y., Ninomiya, H., Miwa, S., and Masaki., T. (2000) J Biol Chem 275(9), 6439-4
32. Bayer, N., SCHOBER, D., PRCHLA, E., MURPHY, R., BLAAS, D., and FUCHS1, R. (1998) JOURNAL OF VIROLOGY December, 9645-9655
33. Catizone, A., Chiantore, M., Andreola, F., Coletti, D., Albani, M., and Alescio, T. (1996) Cell Mol Biol 42(8), 1229-1242
34. Rossi, M., Gorbunoff, M. J., Caruso, F., Wing, B., B, P.-R., and N., T. S. (1996) Biochemistry 35, 3286-3289
35. Adam, S. A., Marr, R. S., and Gerace, L. (1990) J Cell Biol 111, 807
36. Adam, S. A., and Gerace, L. (1991) Cell 66, 837-847
37. Anderson, R. G. W., Kamen, B. A., Rothberg, K. G., and Lacey, S. W. (1998) scienve 255(410-411)
38. Suzuki, T., Futaki, S., Niwa, M., Tanaka, S., Ueda, K., and Sugiura, Y. (2002) J Biol Chem 277(4):2437-43(4), 2437-43
39. Futaki, S., Suzuki, T., Ohashi, W., Yagami, T., Tanaka, S., Ueda, K., and Sugiura, Y. (2000) J. Biol. Chem. 276(8), 5836-40
40. Polyakov, V., Sharma, V., Dahlheimer, J., Pica, C., Luker, G., and PiwnicaWorms, D. (2000) Bioconjugate Chem. 11, 762-771
41. Plank, C., Zauner, W., and Wagner, E. (1998) Adv Drug Del Rev 34, 21-35
42. Efthymiadis, A., Briggs, L. J., and Jans, D. A. (1998) J Biol Chem 273(3), 1623-1628
43. Friedler, A., N. Zakai, O. Karni, D. Friedler, C. Gilon, and A. Loyter (1999) Identification of a nuclear transport inhibitory signal (NTIS) in the basic domains of HIV-1 Vif protein. J Mol Biol 289, 431-437.
44. Karni, O., A. Friedler, N. Zakai, C. Gilon, and A. Loyter (1998) A peptide derived from the N-terminal region of HIV-1 Vpr promotes nuclear import in permeabilized cells: Elucidation of the NLS region of the Vpr. FEBS Lett. 429, 421-425.

What is claimed is:

1. A conjugate comprising a histone moiety covalently linked to a macromolecule-of-interest, said histone moiety being transportable through cell membranes and importable into cell nuclei.

2. The conjugate of claim 1, wherein said macromolecule-of-interest has therapeutic activity.

3. The conjugate of claim 1, wherein said macromolecule-of-interest is a non-marker macromolecule.

4. The conjugate of claim 1, wherein said histone moiety is selected from the group consisting of at least one histone protein and at least one derivative of a histone protein.

5. The conjugate of claim 1, wherein said histone moiety comprises a mixture of at least two histone proteins selected from the group consisting of H1, H2A, H2B, H3 and H4.

6. The conjugate of claim 1, wherein said histone moiety comprises H2A.

7. The conjugate of claim 1, wherein said macromolecule-of-interest is a chemically synthesized macromolecule.

8. The conjugate of claim 1, wherein said macromolecule-of-interest is isolated from a biological source.

9. The conjugate of claim 1, wherein said macromolecule-of-interest is a protein.

10. The conjugate of claim 1, wherein said macromolecule-of-interest is a nucleic acid.

11. The conjugate of claim 10, wherein said nucleic acid is an oligonucleotide.

12. The conjugate of claim 10, wherein said nucleic acid is a DNA.

13. The conjugate of claim 10, wherein said nucleic acid is an RNA.

14. The conjugate of claim 10, wherein said nucleic acid encodes for a gene.

15. The conjugate of claim 1, wherein said histone moiety is covalently linked to said macromolecule-of-interest via a spacer.

16. The conjugate of claim 15, wherein said spacer comprises a sulfide bond.

17. The conjugate of claim 1, wherein said histone moiety is covalently linked to said macromolecule-of-interest via a non-peptide bond.

18. A polynucleotide encoding an in-frame polypeptide conjugate, said polypeptide conjugate comprises a histone moiety and a protein-of-interest, said histone moiety being transportable through cell membranes and importable into cell nuclei.

19. The polynucleotide of claim 18, wherein said protein-of-interest has therapeutic activity.

20. The polynucleotide of claim 18, wherein said protein-of-interest is a non-marker protein.

21. The polynucleotide of claim 18, wherein said histone moiety is selected from the group consisting of H1, H2A, H2B, H3 and H4.

22. The polynucleotide of claim 18, wherein said histone moiety comprises H2A.

23. A nucleic acid construct, comprising the polynucleotide of claim 18.

24. The nucleic acid construct of claim 23, further comprising a cis-acting regulatory element.

25. A pharmaceutical composition comprising, as an active ingredient, the conjugate of claim 1 and a pharmaceutically acceptable carrier.

26. The pharmaceutical composition of claim 25, identified for use in the treatment of a proliferative disorder or disease, a genetic disorder or disease, a bacterial infection or a viral infection.

27. The pharmaceutical composition of claim 25, wherein said macromolecule-of-interest has therapeutic activity.

28. The pharmaceutical composition of claim 25, wherein said macromolecule-of-interest is a non-marker macromolecule.

29. The pharmaceutical composition of claim 25, wherein said histone moiety is selected from the group consisting of at least one histone protein and at least one derivative of a histone protein.

30. The pharmaceutical composition of claim 25, wherein said histone moiety comprises a mixture of at least two histone proteins selected from the group consisting of H1, H2A, H2B, H3 and H4.

31. The pharmaceutical composition of claim 25, wherein said histone moiety comprises H2A.

32. The pharmaceutical composition of claim 25, wherein said macromolecule-of-interest is a chemically synthesized macromolecule.

33. The pharmaceutical composition of claim 25, wherein said macromolecule-of-interest is isolated from a biological source.

34. The pharmaceutical composition of claim 25, wherein said macromolecule-of-interest is a protein.

35. The pharmaceutical composition of claim 25, wherein said macromolecule-of-interest is a nucleic acid.

36. The pharmaceutical composition of claim 35, wherein said nucleic acid is an oligonucleotide.

37. The pharmaceutical composition of claim 35, wherein said nucleic acid is a DNA.

38. The pharmaceutical composition of claim 35, wherein said nucleic acid is an RNA.

39. The pharmaceutical composition of claim 35, wherein said nucleic acid encodes for a gene.

40. The pharmaceutical composition of claim 25, wherein said histone moiety is covalently linked to said macromolecule-of-interest via a spacer.

41. The pharmaceutical composition of claim 40, wherein said spacer comprises a sulfide bond.

42. The pharmaceutical composition of claim 25, wherein said histone moiety is covalently linked to said macromolecule-of-interest via a non-peptide bond.

43. A method of synthesizing the conjugate of claim 1, the method comprising covalently linking said histone moiety and said macromolecule-of-interest, to thereby produce said conjugate.

44. The method of claim 43, further comprising, prior to said covalently linking, covalently attaching a spacer to said macromolecule-of-interest.

45. The method of claim 43, further comprising, prior to said covalently linking, covalently attaching a spacer to said histone moiety.

46. The method of claim 44, further comprising, prior to said covalently linking, functionalizing said histone moiety into a functionalized derivative which comprises a free functional group.

47. The method of claim 46, wherein covalently linking said histone moiety and said macromolecule-of-interest comprises covalently attaching said functional group to said spacer.

48. The method of claim 46, wherein said functionalized derivative is a thiolated derivative and said functional group is a thiol group.

49. The method of claim 45, further comprising, prior to said covalently linking, functionalizing said macromolecule-of-interest into a functionalized derivative which comprises a free functional group.

50. The method of claim 49, wherein said functionalized derivative is a thiolated derivative and said functional group is a thiol group.

51. The method of claim 49, wherein covalently linking said histone moiety and said macromolecule-of-interest comprises covalently attaching said functional group to said spacer.

52. The method of claim 43, wherein said covalently linking is performed using a cross-linking agent.

53. The method of claim 52, wherein said cross-linking agent is Sulfo-SMMC.

54. The method of claim 43, wherein said macromolecule-of-interest has therapeutic activity.

55. The method of claim 43, wherein said macromolecule-of-interest is a non-marker macromolecule.

56. The method of claim 43, wherein said histone moiety is selected from the group consisting of at least one histone protein and at least one derivative of a histone protein.

57. The method of claim 43, wherein said histone moiety comprises a mixture of at least two histone proteins selected from the group consisting of H1, H2A, H2B, H3 and H4.

58. The method of claim 43, wherein said histone moiety comprises H2A.

59. The method of claim 43, wherein said macromolecule-of-interest is a chemically synthesized macromolecule.

60. The method of claim 43, wherein said macromolecule-of-interest is isolated from a biological source.

61. The method of claim 43, wherein said macromolecule-of-interest is a protein.

62. The method of claim 43, wherein said macromolecule-of-interest is a nucleic acid.

63. The method of claim 62, wherein said nucleic acid is an oligonucleotide.

64. The method of claim 62, wherein said nucleic acid is a DNA.

65. The method of claim 62, wherein said nucleic acid is an RNA.

66. The method of claim 62, wherein said nucleic acid encodes for a gene.

67. The method of claim 43, wherein said histone moiety is covalently linked to said macromolecule-of-interest via a spacer.

68. The method of claim 67, wherein said spacer comprises a sulfide bond.

69. A method of delivering a macromolecule-of-interest into a cell, the method comprising contacting the cell with the conjugate of claim 1.

70. The method of claim 69, wherein said contacting is performed by co-incubating said cell and said conjugate.

71. The method of claim 69, wherein said macromolecule-of-interest has therapeutic activity.

72. The method of claim 69, wherein said macromolecule-of-interest is a non-marker macromolecule.

73. The method of claim 69, wherein said histone moiety is selected from the group consisting of at least one histone protein and at least one derivative of a histone protein.

74. The method of claim 69, wherein said histone moiety comprises a mixture of at least two histone proteins selected from the group consisting of H1, H2A, H2B, H3 and H4.

75. The method of claim 69, wherein said at least one histone moiety comprises H2A.

76. The method of claim 69, wherein said macromolecule-of-interest is a chemically synthesized macromolecule.

77. The method of claim 69, wherein said macromolecule-of-interest is isolated from a biological source.

78. The method of claim 69, wherein said macromolecule-of-interest is a protein.

79. The method of claim 69, wherein said macromolecule-of-interest is a nucleic acid.

80. The method of claim 79, wherein said nucleic acid is an oligonucleotide.

81. The method of claim 79, wherein said nucleic acid is a DNA.

82. The method of claim 79, wherein said nucleic acid is an RNA.

83. The method of claim 79, wherein said nucleic acid encodes for a gene.

84. The method of claim 69, wherein said histone moiety is covalently linked to said macromolecule-of-interest via a spacer.

85. The method of claim 84, wherein said spacer comprises a sulfide bond.

86. The method of claim 69, wherein said histone moiety is covalently linked to said macromolecule-of-interest via a non-peptide bond.

87. A method of treating a proliferative disorder or disease, a genetic disorder or disease, a bacterial infection and/or a viral infection in a subject in need thereof, the method comprising administering to the subject a therapeutically effective amount of the conjugate of claim 1, wherein said macromolecule-of-interest has a therapeutic activity in treating said proliferative disorder or disease, said genetic disorder or disease, said bacterial infection and/or said viral infection.

88. The method of claim 87, wherein said histone moiety is selected from the group consisting of at least one histone protein and at least one derivative of a histone protein.

89. The method of claim 87, wherein said histone moiety comprises a mixture of at least two histone proteins selected from the group consisting of H1, H2A, H2B, H3 and H4.

90. The method of claim 87, wherein said at least one histone moiety comprises H2A.

91. The method of claim 87, wherein said macromolecule-of-interest is a chemically synthesized macromolecule.

92. The method of claim 87, wherein said macromolecule-of-interest is isolated from a biological source.

93. The method of claim 87, wherein said macromolecule-of-interest is a protein.

94. The method of claim 87, wherein said macromolecule-of-interest is a nucleic acid.

95. The method of claim 94, wherein said nucleic acid is an oligonucleotide.

96. The method of claim 94, wherein said nucleic acid is a DNA.

97. The method of claim 94, wherein said nucleic acid is an RNA.

98. The method of claim 94, wherein said nucleic acid encodes for a gene.

99. The method of claim 87, wherein said histone moiety is covalently linked to said macromolecule-of-interest via a spacer.

100. The method of claim 99, wherein said spacer comprises a sulfide bond.

101. The method of claim 87, wherein said histone moiety is covalently linked to said macromolecule of interest via a non-peptide bond.

102. A method of quantitatively determining a nuclear uptake and/or a cytoplasmic uptake of a moiety into cells, the method comprising:
contacting said moiety with said cells;
fractionating said cells into a cytoplasmic fraction and a nuclei fraction; and
quantitatively determining an amount or concentration of said moiety in said cytoplasmic fraction and in said nuclei fraction, thereby quantitatively determining the nuclear uptake and/or the cytoplasmic uptake of the moiety into the cells.

103. The method of claim 102, wherein said contacting is performed by co-incubating said cells and said moiety.

104. The method of claim 102, wherein said fractionating is performed by permeabilizing the plasma membrane of said cells, to thereby obtain said cytoplasmic fraction and thereafter permeabilizing the nuclear membrane of said cells, to thereby obtain said nuclei fraction.

105. The method of claim 102, wherein said quantitatively determining comprises:
contacting said cytoplasmic fraction or said nuclei fraction with a solid phase having binding affinity to said moiety, to thereby adhere said moiety to said solid phase;
affinity attaching a detectable molecule to said moiety; and
quantitatively detecting an amount or concentration of said detectable molecule affinity bound to said moiety, to thereby quantitatively determining the amount or concentration of said moiety in said cytoplasmic fraction or in said nuclei fraction.

106. The method of claim 105, wherein said solid phase is selected from the group consisting of a microtiter plate, a chip and a glass.

107. The method of claim 105, wherein said detectable molecule comprises an enzyme capable of catalyzing a colorimetric reaction, a bead, a pigment and a fluorophore.

108. The method of claim 102, wherein said moiety includes a detection group attached thereto.

109. The method of claim 108, wherein said detection group is biotin.

110. The method of claim 102, wherein said moiety is a macromolecule.

111. The method of claim 110, wherein said macromolecule is a protein.

112. The method of claim 110, wherein said macromolecule is a nucleic acid.

113. The method of claim 110, wherein said macromolecule is a histone moiety.

114. The method of claim 113, wherein said histone moiety is selected from the group consisting of at least one histone protein and at least one derivative of a histone protein.

115. The method of claim 113, wherein said histone moiety comprises a mixture of at least two histone proteins selected from the group consisting of H1, H2A, H2B, H3 and H4.

116. The method of claim 113, wherein said histone moiety comprises H2A.

117. The method of claim 110, wherein said macromolecule is a chemically synthesized macromolecule.

118. The method of claim 110, wherein said macromolecule is isolated from a biological source.

119. The method of claim 112, wherein said nucleic acid is an oligonucleotide.

120. The method of claim 112, wherein said nucleic acid is a DNA.

121. The method of claim 112, wherein said nucleic acid is an RNA.

122. The method of claim 112, wherein said nucleic acid encodes for a gene.

123. The method of claim 102, wherein said moiety is a conjugate of a first macromolecule covalently attached to a second macromolecule.

124. The method of claim 123, wherein said first macromolecule is a histone moiety and said second macromolecule is selected from the group consisting of a protein and a nucleic acid.

125. The method of claim 124, wherein said histone moiety is selected from the group consisting of at least one histone protein and at least one derivative of a histone protein.

126. The method of claim 124, wherein said histone moiety comprises a mixture of at least two histone proteins selected from the group consisting of H1, H2A, H2B, H3 and H4.

127. The method of claim 124, wherein said histone moiety comprises H2A.

128. The method of claim 124, wherein said nucleic acid is an oligonucleotide.

129. The method of claim 124, wherein said nucleic acid is a DNA.

130. The method of claim 124, wherein said nucleic acid is an RNA.

131. The method of claim 124, wherein said nucleic acid encodes for a gene.
